SEQUENCE LISTING (144 sequences)

SEQ ID NO: 1 - protein, 12 aa, Artificial Sequence
  synthetic
CEESFYPECDHC

SEQ ID NO: 2 - protein, 12 aa, Artificial Sequence
  synthetic
CPDLCLDLFPNC

SEQ ID NO: 3 - protein, 12 aa, Artificial Sequence
  synthetic
CPELCVDLYPHC

SEQ ID NO: 4 - protein, 12 aa, Artificial Sequence
  synthetic
CHPEWVSCEFHC

SEQ ID NO: 5 - protein, 13 aa, Artificial Sequence
  synthetic
CSHECALLFPKTC

SEQ ID NO: 6 - protein, 13 aa, Artificial Sequence
  synthetic
CFDECQLLFPKTC

SEQ ID NO: 7 - protein, 13 aa, Artificial Sequence
  synthetic
CLDECKLLFPKTC

SEQ ID NO: 8 - protein, 13 aa, Artificial Sequence
  synthetic
CREECMLLFPKTC

SEQ ID NO: 9 - protein, 13 aa, Artificial Sequence
  synthetic
CETECALLFPRSC

SEQ ID NO: 10 - protein, 13 aa, Artificial Sequence
  synthetic
CADECRLLFPKTC

SEQ ID NO: 11 - protein, 13 aa, Artificial Sequence
  synthetic
CDVECRLLFPRSC

SEQ ID NO: 12 - protein, 13 aa, Artificial Sequence
  synthetic
CIDECRLLFPRSC

SEQ ID NO: 13 - protein, 13 aa, Artificial Sequence
  synthetic
CVRECALLFPKTC

SEQ ID NO: 14 - protein, 13 aa, Artificial Sequence
  synthetic
  MISC_FEATURE (3)..(3): Xaa represents HArg
CVXECALLFPKTC

SEQ ID NO: 15 - protein, 13 aa, Artificial Sequence
  synthetic
CVRECALLFPRTC

SEQ ID NO: 16 - protein, 13 aa, Artificial Sequence
  synthetic
  MISC_FEATURE (11)..(11): Xaa represents HArg
CVRECALLFPXTC

SEQ ID NO: 17 - protein, 13 aa, Artificial Sequence
  synthetic
  MISC_FEATURE (3)..(3): Xaa represents HArg
  MISC_FEATURE (11)..(11): Xaa represents HArg
CVXECALLFPXTC

SEQ ID NO: 18 - protein, 13 aa, Artificial Sequence
  synthetic
  MISC_FEATURE (3)..(3): Xaa represents HArg
CVXECALLFPATC

SEQ ID NO: 19 - protein, 13 aa, Artificial Sequence
  synthetic
  MISC_FEATURE (11)..(11): Xaa represents HArg
CVAECALLFPXTC

SEQ ID NO: 20 - protein, 13 aa, Artificial Sequence
  synthetic
CVTECQLLFPKTC

SEQ ID NO: 21 - protein, 13 aa, Artificial Sequence
  synthetic
CRHECELLFPKTC

SEQ ID NO: 22 - protein, 13 aa, Artificial Sequence
  synthetic
CQRECALLFPKTC

SEQ ID NO: 23 - protein, 13 aa, Artificial Sequence
  synthetic
CVRECTLLFPKTC

SEQ ID NO: 24 - protein, 13 aa, Artificial Sequence
  synthetic
CTIECALLFPKTC

SEQ ID NO: 25 - protein, 13 aa, Artificial Sequence
  synthetic
CARECALLFPKTC

SEQ ID NO: 26 - protein, 13 aa, Artificial Sequence
  synthetic
CINECRLLFPKTC

SEQ ID NO: 27 - protein, 13 aa, Artificial Sequence
  synthetic
CYTECSLLFPKTC

SEQ ID NO: 28 - protein, 13 aa, Artificial Sequence
  synthetic
CHEECRLLFPKTC

SEQ ID NO: 29 - protein, 13 aa, Artificial Sequence
  synthetic
CLEECKLLFPKTC

SEQ ID NO: 30 - protein, 13 aa, Artificial Sequence
  synthetic
CIDECALLFPRTC

SEQ ID NO: 31 - protein, 13 aa, Artificial Sequence
  synthetic
CYEECRLLFPRTC

SEQ ID NO: 32 - protein, 13 aa, Artificial Sequence
  synthetic
CVRECRLLFPKTC

SEQ ID NO: 33 - protein, 13 aa, Artificial Sequence
  synthetic
CHIECALLFPKTC

SEQ ID NO: 34 - protein, 13 aa, Artificial Sequence
  synthetic
CKRECMLLFPKTC

SEQ ID NO: 35 - protein, 13 aa, Artificial Sequence
  synthetic
CYRECALLFPKTC

SEQ ID NO: 36 - protein, 13 aa, Artificial Sequence
  synthetic
CLTECALLFPKTC

SEQ ID NO: 37 - protein, 13 aa, Artificial Sequence
  synthetic
CEVECRLLFPKTC

SEQ ID NO: 38 - protein, 13 aa, Artificial Sequence
  synthetic
CEAECRLLFPKTC

SEQ ID NO: 39 - protein, 13 aa, Artificial Sequence
  synthetic
CVQECALLFPKTC

SEQ ID NO: 40 - protein, 13 aa, Artificial Sequence
  synthetic
CIRECSLLFPKTC

SEQ ID NO: 41 - protein, 13 aa, Artificial Sequence
  synthetic
CVTECALLFPKTC

SEQ ID NO: 42 - protein, 13 aa, Artificial Sequence
  synthetic
CVAECKLLFPKTC

SEQ ID NO: 43 - protein, 13 aa, Artificial Sequence
  synthetic
CVGECALLFPKTC

SEQ ID NO: 44 - protein, 13 aa, Artificial Sequence
  synthetic
CVVECALLFPKTC

SEQ ID NO: 45 - protein, 13 aa, Artificial Sequence
  synthetic
CVFECALLFPKTC

SEQ ID NO: 46 - protein, 13 aa, Artificial Sequence
  synthetic
  MISC_FEATURE (3)..(3): Xaa represents HArg
  MISC_FEATURE (11)..(11): Xaa represents HArg
CAXECALLFPXTC

SEQ ID NO: 47 - protein, 13 aa, Artificial Sequence
  synthetic
  MISC_FEATURE (3)..(3): Xaa represents HArg
  MISC_FEATURE (11)..(11): Xaa represents HArg
CVXECALLFAXTC

SEQ ID NO: 48 - protein, 13 aa, Artificial Sequence
  synthetic
  MISC_FEATURE (3)..(3): Xaa represents HArg
  MISC_FEATURE (11)..(11): Xaa represents HArg
CVXECALLFPXAC

SEQ ID NO: 49 - protein, 13 aa, Artificial Sequence
  synthetic
  MISC_FEATURE (3)..(3): Xaa represents HArg
  MISC_FEATURE (9)..(9): Xaa represents 1Nal
  MISC_FEATURE (11)..(11): Xaa represents HArg
CVXECALLXPXTC

SEQ ID NO: 50 - protein, 13 aa, Artificial Sequence
  synthetic
  MISC_FEATURE (3)..(3): Xaa represents HArg
  MISC_FEATURE (9)..(9): Xaa represents Cha
  MISC_FEATURE (11)..(11): Xaa represents HArg
CVXECALLXPXTC

SEQ ID NO: 51 - protein, 13 aa, Artificial Sequence
  synthetic
  MISC_FEATURE (3)..(3): Xaa represents HArg
  MISC_FEATURE (10)..(10): Xaa represents Pip
  MISC_FEATURE (11)..(11): Xaa represents HArg
CVXECALLFXXTC

SEQ ID NO: 52 - protein, 13 aa, Artificial Sequence
  synthetic
  MISC_FEATURE (3)..(3): Xaa represents HArg
  MISC_FEATURE (11)..(11): Xaa represents HArg
CVXECALLFPXSC

SEQ ID NO: 53 - protein, 13 aa, Artificial Sequence
  synthetic
  MISC_FEATURE (3)..(3): Xaa represents HArg
  MISC_FEATURE (11)..(11): Xaa represents HArg
  MISC_FEATURE (12)..(12): Xaa represents HSer
CVXECALLFPXXC

SEQ ID NO: 54 - protein, 13 aa, Artificial Sequence
  synthetic
  MISC_FEATURE (3)..(3): Xaa represents HArg
  MISC_FEATURE (10)..(10): Xaa represents HyP
  MISC_FEATURE (11)..(11): Xaa represents HArg
CVXECALLFXXTC

SEQ ID NO: 55 - protein, 13 aa, Artificial Sequence
  synthetic
  MISC_FEATURE (3)..(3): Xaa represents HArg
  MISC_FEATURE (6)..(6): Xaa represents Aib
  MISC_FEATURE (11)..(11): Xaa represents HArg
CVXECXLLFPXTC

SEQ ID NO: 56 - protein, 13 aa, Artificial Sequence
  synthetic
  MISC_FEATURE (3)..(3): Xaa represents HArg
  MISC_FEATURE (8)..(8): Xaa represents Nle
  MISC_FEATURE (11)..(11): Xaa represents HArg
CVXECALXFPXTC

SEQ ID NO: 57 - protein, 13 aa, Artificial Sequence
  synthetic
  MISC_FEATURE (3)..(3): Xaa represents HArg
  MISC_FEATURE (7)..(7): Xaa represents tBuAla
  MISC_FEATURE (11)..(11): Xaa represents HArg
CVXECAXLFPXTC

SEQ ID NO: 58 - protein, 13 aa, Artificial Sequence
  synthetic
  MISC_FEATURE (3)..(3): Xaa represents HArg
  MISC_FEATURE (7)..(7): Xaa represents Nle
  MISC_FEATURE (11)..(11): Xaa represents HArg
CVXECAXLFPXTC

SEQ ID NO: 59 - protein, 13 aa, Artificial Sequence
  synthetic
  MISC_FEATURE (3)..(3): Xaa represents Aad2
  MISC_FEATURE (11)..(11): Xaa represents HArg
CVXECALLFPXTC

SEQ ID NO: 60 - protein, 13 aa, Artificial Sequence
  synthetic
  MISC_FEATURE (3)..(3): Xaa represents HArg
  MISC_FEATURE (11)..(11): Xaa represents HArg
CPXECALLFPXTC

SEQ ID NO: 61 - protein, 13 aa, Artificial Sequence
  synthetic
  MISC_FEATURE (3)..(3): Xaa represents HArg
  MISC_FEATURE (9)..(9): Xaa represents 4FlPhe
  MISC_FEATURE (11)..(11): Xaa represents HArg
CVXECALLXPXTC

SEQ ID NO: 62 - protein, 13 aa, Artificial Sequence
  synthetic
  MISC_FEATURE (3)..(3): Xaa represents HArg
  MISC_FEATURE (8)..(8): Xaa represents tBuGly
  MISC_FEATURE (11)..(11): Xaa represents HArg
CVXECALXFPXTC

SEQ ID NO: 63 - protein, 13 aa, Artificial Sequence
  synthetic
  MISC_FEATURE (3)..(3): Xaa represents HArg
  MISC_FEATURE (8)..(8): Xaa represents Cha
  MISC_FEATURE (11)..(11): Xaa represents HArg
CVXECALXFPXTC

SEQ ID NO: 64 - protein, 13 aa, Artificial Sequence
  synthetic
  MISC_FEATURE (3)..(3): Xaa represents HArg
  MISC_FEATURE (9)..(9): Xaa represents 2Nal
  MISC_FEATURE (11)..(11): Xaa represents HArg
CVXECALLXPXTC

SEQ ID NO: 65 - protein, 13 aa, Artificial Sequence
  synthetic
  MISC_FEATURE (3)..(3): Xaa represents HArg
  MISC_FEATURE (11)..(11): Xaa represents HArg
  MISC_FEATURE (12)..(12): Xaa represents HyV
CVXECALLFPXXC

SEQ ID NO: 66 - protein, 13 aa, Artificial Sequence
  synthetic
  MISC_FEATURE (2)..(2): Xaa represents tBuGly
  MISC_FEATURE (3)..(3): Xaa represents HArg
  MISC_FEATURE (11)..(11): Xaa represents HArg
CXXECALLFPXTC

SEQ ID NO: 67 - protein, 13 aa, Artificial Sequence
  synthetic
  MISC_FEATURE (11)..(11): Xaa represents HArg
CVEECALLFPXTC

SEQ ID NO: 68 - protein, 13 aa, Artificial Sequence
  synthetic
  MISC_FEATURE (3)..(3): Xaa represents HArg
  MISC_FEATURE (7)..(7): Xaa represents Cpa
  MISC_FEATURE (11)..(11): Xaa represents HArg
CVXECAXLFPXTC

SEQ ID NO: 69 - protein, 13 aa, Artificial Sequence
  synthetic
  MISC_FEATURE (3)..(3): Xaa represents HArg
  MISC_FEATURE (7)..(7): Xaa represents Cba
  MISC_FEATURE (11)..(11): Xaa represents HArg
CVXECAXLFPXTC

SEQ ID NO: 70 - protein, 13 aa, Artificial Sequence
  synthetic
  MISC_FEATURE (3)..(3): Xaa represents HArg
  MISC_FEATURE (7)..(7): Xaa represents C5A
  MISC_FEATURE (11)..(11): Xaa represents HArg
CVXECAXLFPXTC

SEQ ID NO: 71 - protein, 13 aa, Artificial Sequence
  synthetic
  MISC_FEATURE (3)..(3): Xaa represents HArg
  MISC_FEATURE (7)..(7): Xaa represents Cha
  MISC_FEATURE (11)..(11): Xaa represents HArg
CVXECAXLFPXTC

SEQ ID NO: 72 - protein, 13 aa, Artificial Sequence
  synthetic
  MISC_FEATURE (3)..(3): Xaa represents HArg
  MISC_FEATURE (7)..(7): Xaa represents tBuGly
  MISC_FEATURE (11)..(11): Xaa represents HArg
CVXECAXLFPXTC

SEQ ID NO: 73 - protein, 13 aa, Artificial Sequence
  synthetic
  MISC_FEATURE (3)..(3): Xaa represents HArg
  MISC_FEATURE (10)..(10): Xaa represents cis-HyP
  MISC_FEATURE (11)..(11): Xaa represents HArg
CVXECALLFXXTC

SEQ ID NO: 74 - protein, 13 aa, Artificial Sequence
  synthetic
  MISC_FEATURE (3)..(3): Xaa represents HArg
  MISC_FEATURE (8)..(8): Xaa represents Cpa
  MISC_FEATURE (11)..(11): Xaa represents HArg
CVXECALXFPXTC

SEQ ID NO: 75 - protein, 13 aa, Artificial Sequence
  synthetic
  MISC_FEATURE (3)..(3): Xaa represents HArg
  MISC_FEATURE (8)..(8): Xaa represents C5A
  MISC_FEATURE (11)..(11): Xaa represents HArg
CVXECALXFPXTC

SEQ ID NO: 76 - protein, 13 aa, Artificial Sequence
  synthetic
  MISC_FEATURE (3)..(3): Xaa represents HArg
  MISC_FEATURE (7)..(7): Xaa represents tBuAla
  MISC_FEATURE (10)..(10): Xaa represents HyP
  MISC_FEATURE (11)..(11): Xaa represents HArg
CVXECAXLFXXTC

SEQ ID NO: 77 - protein, 13 aa, Artificial Sequence
  synthetic
  MISC_FEATURE (3)..(3): Xaa represents HArg
  MISC_FEATURE (7)..(7): Xaa represents tBuAla
  MISC_FEATURE (8)..(8): Xaa represents tBuGly
  MISC_FEATURE (10)..(10): Xaa represents HyP
  MISC_FEATURE (11)..(11): Xaa represents HArg
CVXECAXXFXXTC

SEQ ID NO: 78 - protein, 13 aa, Artificial Sequence
  synthetic
  MISC_FEATURE (2)..(2): Xaa represents tBuGly
  MISC_FEATURE (3)..(3): Xaa represents HArg
  MISC_FEATURE (7)..(7): Xaa represents tBuAla
  MISC_FEATURE (11)..(11): Xaa represents HArg
CXXECAXLFPXTC

SEQ ID NO: 79 - protein, 13 aa, Artificial Sequence
  synthetic
CSSWDKLMCHPYC

SEQ ID NO: 80 - protein, 15 aa, Artificial Sequence
  synthetic
CPEECFYLPPHPMSC

SEQ ID NO: 81 - protein, 15 aa, Artificial Sequence
  synthetic
CPQECFYLPGHSLYC

SEQ ID NO: 82 - protein, 15 aa, Artificial Sequence
  synthetic
CPGECFYPPGHPLAC

SEQ ID NO: 83 - protein, 15 aa, Artificial Sequence
  synthetic
CPGECFYPTNHPLYC

SEQ ID NO: 84 - protein, 15 aa, Artificial Sequence
  synthetic
CPQECFYPIGHPLAC

SEQ ID NO: 85 - protein, 15 aa, Artificial Sequence
  synthetic
CPEECFYPPGHKLHC

SEQ ID NO: 86 - protein, 15 aa, Artificial Sequence
  synthetic
CPQECFYPPGHRLRC

SEQ ID NO: 87 - protein, 15 aa, Artificial Sequence
  synthetic
CPQECFYPPGHPYHC

SEQ ID NO: 88 - protein, 15 aa, Artificial Sequence
  synthetic
CPQECFYPSTHPLYC

SEQ ID NO: 89 - protein, 15 aa, Artificial Sequence
  synthetic
CPGECFYPSNHRLYC

SEQ ID NO: 90 - protein, 15 aa, Artificial Sequence
  synthetic
CPDECFYPPEHPLAC

SEQ ID NO: 91 - protein, 15 aa, Artificial Sequence
  synthetic
CPGECFYPPGHHLSC

SEQ ID NO: 92 - protein, 15 aa, Artificial Sequence
  synthetic
CPGECFYPPGHHLGC

SEQ ID NO: 93 - protein, 15 aa, Artificial Sequence
  synthetic
CPEECFYPPNHPLYC

SEQ ID NO: 94 - protein, 15 aa, Artificial Sequence
  synthetic
CPGECFYPPDHPLYC

SEQ ID NO: 95 - protein, 15 aa, Artificial Sequence
  synthetic
CPGECFYPPGHPLYC

SEQ ID NO: 96 - protein, 15 aa, Artificial Sequence
  synthetic
CPGECFYPPNHPFYC

SEQ ID NO: 97 - protein, 15 aa, Artificial Sequence
  synthetic
CPGECFYPPNHPLYC

SEQ ID NO: 98 - protein, 15 aa, Artificial Sequence
  synthetic
CPEECFYPPGHPLAC

SEQ ID NO: 99 - protein, 15 aa, Artificial Sequence
  synthetic
CWMECFYPPGHPLAC

SEQ ID NO: 100 - protein, 15 aa, Artificial Sequence
  synthetic
CFEECFYPPGHPLAC

SEQ ID NO: 101 - protein, 15 aa, Artificial Sequence
  synthetic
CPGECFYPPGHPLRC

SEQ ID NO: 102 - protein, 15 aa, Artificial Sequence
  synthetic
CPGECFYPPGHPREC

SEQ ID NO: 103 - protein, 15 aa, Artificial Sequence
  synthetic
CPGECFYPPGHRFHC

SEQ ID NO: 104 - protein, 15 aa, Artificial Sequence
  synthetic
CPGECFYPPGHRLYC

SEQ ID NO: 105 - protein, 15 aa, Artificial Sequence
  synthetic
CEEEFYPCGHPLYVC

SEQ ID NO: 106 - protein, 15 aa, Artificial Sequence
  synthetic
CEEQFYPCTHALYTC

SEQ ID NO: 107 - protein, 15 aa, Artificial Sequence
  synthetic
CVEEFYPCDHPLYSC

SEQ ID NO: 108 - protein, 15 aa, Artificial Sequence
  synthetic
CEEEFYPCGHPMHPC

SEQ ID NO: 109 - protein, 15 aa, Artificial Sequence
  synthetic
CDEQFYPCHHRLYSC

SEQ ID NO: 110 - protein, 15 aa, Artificial Sequence
  synthetic
CEEEFYPCGHPFHPC

SEQ ID NO: 111 - protein, 15 aa, Artificial Sequence
  synthetic
CLEQFYPCEHPLFSC

SEQ ID NO: 112 - protein, 15 aa, Artificial Sequence
  synthetic
CVEQFYPCGHRHYIC

SEQ ID NO: 113 - protein, 15 aa, Artificial Sequence
  synthetic
CEEQFYPCSHPLYTC

SEQ ID NO: 114 - protein, 15 aa, Artificial Sequence
  synthetic
CEEQFYPCNHPLNVC

SEQ ID NO: 115 - protein, 15 aa, Artificial Sequence
  synthetic
CEEEFYPCSHPLNPC

SEQ ID NO: 116 - protein, 15 aa, Artificial Sequence
  synthetic
CEEQFYPCGHKLSPC

SEQ ID NO: 117 - protein, 15 aa, Artificial Sequence
  synthetic
CPEQFYPCDHRLYIC

SEQ ID NO: 118 - protein, 15 aa, Artificial Sequence
  synthetic
CQEQFYPCNHPLSPC

SEQ ID NO: 119 - protein, 15 aa, Artificial Sequence
  synthetic
CDEQFYPCNHRLNTC

SEQ ID NO: 120 - protein, 15 aa, Artificial Sequence
  synthetic
CEEAFYPCHHPLYRC

SEQ ID NO: 121 - protein, 15 aa, Artificial Sequence
  synthetic
CDEDFYPCGHYLNQC

SEQ ID NO: 122 - protein, 15 aa, Artificial Sequence
  synthetic
CEEQFYPCTHPLYVC

SEQ ID NO: 123 - protein, 15 aa, Artificial Sequence
  synthetic
CPEQFYPCTHRLYQC

SEQ ID NO: 124 - protein, 15 aa, Artificial Sequence
  synthetic
CEEQFYPCSHPLYRC

SEQ ID NO: 125 - protein, 15 aa, Artificial Sequence
  synthetic
CAEQFYPCDHPLYRC

SEQ ID NO: 126 - protein, 15 aa, Artificial Sequence
  synthetic
CAEEFYPCDHPLYRC

SEQ ID NO: 127 - protein, 15 aa, Artificial Sequence
  synthetic
CEEAFYPCNHPLYTC

SEQ ID NO: 128 - protein, 15 aa, Artificial Sequence
  synthetic
CAEAFYPCDHPLYVC

SEQ ID NO: 129 - protein, 15 aa, Artificial Sequence
  synthetic
CEEAFYPCSHPLFIC

SEQ ID NO: 130 - protein, 15 aa, Artificial Sequence
  synthetic
CEEAFYPCSHPLHPC

SEQ ID NO: 131 - protein, 15 aa, Artificial Sequence
  synthetic
CEEAFYPCSHPLFVC

SEQ ID NO: 132 - protein, 15 aa, Artificial Sequence
  synthetic
CEEQFYPCSHPLYSC

SEQ ID NO: 133 - protein, 15 aa, Artificial Sequence
  synthetic
CEEAFYPCEHPLYMC

SEQ ID NO: 134 - protein, 15 aa, Artificial Sequence
  synthetic
CEEQFYPCNHPLYMC

SEQ ID NO: 135 - protein, 14 aa, Artificial Sequence
  synthetic
CLEQFYPCGDPRLC

SEQ ID NO: 136 - protein, 14 aa, Artificial Sequence
  synthetic
CEEQFYPCGHHLLC

SEQ ID NO: 137 - protein, 13 aa, Artificial Sequence
  synthetic
CLEPDECFYPMEC

SEQ ID NO: 138 - protein, 13 aa, Artificial Sequence
  synthetic
CKEPQECFYPLKC

SEQ ID NO: 139 - protein, 13 aa, Artificial Sequence
  synthetic
CDSPEECFYPLEC

SEQ ID NO: 140 - protein, 16 aa, Artificial Sequence
  synthetic
ACYNEFGCEDFYDICA

SEQ ID NO: 141 - protein, 16 aa, Artificial Sequence
  synthetic
ACMNQFGCEDFYDICA

SEQ ID NO: 142 - protein, 16 aa, Artificial Sequence
  synthetic
ACYNEFACEDFYDICA

SEQ ID NO: 143 - protein, 17 aa, Artificial Sequence
  synthetic
ACPYSWETCLFGDYRCA

SEQ ID NO: 144 - protein, 17 aa, Artificial Sequence
  synthetic
ACPYDWATCLFGDYRCA

(19) United States
(12) Patent Application Publication
(10) Pub. No.: US 2022/0024982 A1
(43) Pub. Date: Jan. 27, 2022

(54) BICYCLIC PEPTIDE LIGANDS SPECIFIC FOR MT1-MMP

(71) Applicant: BicycleTx Limited, Cambridge (GB)

(72) Inventors: Liuhong CHEN, Cambridge (GB); Euan RICHARDS, Cambridge (GB); Rachid LANI, Cambridge (GB); Gemma MUDD, Cambridge (GB); Catherine STACE, Cambridge (GB); Daniel TEUFEL, Cambridge (GB); Edward WALKER, Cambridge (GB)

(21) Appl. No.: 17/309,626

(22) PCT Filed: Dec. 13, 2019
(86) PCT No.: PCT/GB2019/053540

§ 371 Date: Jun. 11, 2021

(30) Foreign Application Priority Data
Dec. 13, 2018 (GB) 1820286.1
May 9, 2019 (GB) 1906534.1

(51) Int. Cl. C07K 7/64; A61K 47/64

(52) U.S. Cl. CPC C07K 7/64; A61K 47/64

(57) ABSTRACT

The present invention relates to polypeptides which are covalently bound to molecular scaffolds such that two or more peptide loops are subtended between attachment points to the scaffold. In particular, the invention describes peptides which are high affinity binders of membrane type 1 metalloprotease (MT1-MMP). The invention also describes drug conjugates comprising said peptides, conjugated to one or more effector and/or functional groups which have utility in imaging and targeted cancer therapy.

### FIELD OF THE INVENTION

The present invention relates to polypeptides which are covalently bound to molecular scaffolds such that two or more peptide loops are subtended between attachment points to the scaffold. In particular, the invention describes peptides which are high affinity binders of membrane type 1 metalloprotease (MT1-MMP). The invention also describes drug conjugates comprising said peptides, conjugated to one or more effector and/or functional groups which have utility in imaging and targeted cancer therapy.

### BACKGROUND OF THE INVENTION

Cyclic peptides are able to bind with high affinity and target specificity to protein targets and hence are an attractive molecule class for the development of therapeutics. In fact, several cyclic peptides are already successfully used in the clinic, as for example the antibacterial peptide vancomycin, the immunosuppressant drug cyclosporine or the anti-cancer drug octreotide (Driggers et al. (2008), Nat Rev Drug Discov 7 (7), 608-24). Good binding properties result from a relatively large interaction surface formed between the peptide and the target as well as the reduced conformational flexibility of the cyclic structures. Typically, macrocycles bind to surfaces of several hundred square angstrom, as for example the cyclic peptide CXCR4 antagonist CVX15 (400 Å²; Wu et al. (2007), Science 330, 1066-71), a cyclic peptide with the Arg-Gly-Asp motif binding to integrin αVb3 (355 Å²) (Xiong et al. (2002), Science 296 (5565), 151-5) or the cyclic peptide inhibitor upain-1 binding to urokinase-type plasminogen activator (603 Å²; Zhao et al. (2007), J Struct Biol 160 (1), 1-10).

Due to their cyclic configuration, peptide macrocycles are less flexible than linear peptides, leading to a smaller loss of entropy upon binding to targets and resulting in a higher binding affinity. The reduced flexibility also leads to locking target-specific conformations, increasing binding specificity compared to linear peptides. This effect has been exemplified by a potent and selective inhibitor of matrix metalloproteinase 8 (MMP-8) which lost its selectivity over other MMPs when its ring was opened (Cherney et al. (1998), J Med Chem 41 (11), 1749-51). The favorable binding properties achieved through macrocyclization are even more pronounced in multicyclic peptides having more than one peptide ring as for example in vancomycin, nisin and actinomycin.

Different research teams have previously tethered polypeptides with cysteine residues to a synthetic molecular structure (Kemp and McNamara (1985), J. Org. Chem; Timmerman et al. (2005), ChemBioChem). Meloen and co-workers had used tris(bromomethyl)benzene and related molecules for rapid and quantitative cyclisation of multiple peptide loops onto synthetic scaffolds for structural mimicry of protein surfaces (Timmerman et al. (2005), ChemBioChem). Methods for the generation of candidate drug compounds wherein said compounds are generated by linking cysteine containing polypeptides to a molecular scaffold as for example tris(bromomethyl)benzene are disclosed in WO 2004/077062 and WO 2006/078161. Further suitable examples of molecular scaffolds include the non-aromatic scaffolds described in Heinis et al (2014) Angewandte Chemie, International Edition 53(6) 1602-1606.

Phage display-based combinatorial approaches have been developed to generate and screen large libraries of bicyclic peptides to targets of interest (Heinis et al. (2009), Nat Chem Biol 5 (7), 502-7 and WO 2009/098450). Briefly, combinatorial libraries of linear peptides containing three cysteine residues and two regions of six random amino acids (Cys-(Xaa)₆-Cys-(Xaa)₆-Cys) were displayed on phage and cyclised by covalently linking the cysteine side chains to a small molecule scaffold.

### SUMMARY OF THE INVENTION

According to a first aspect of the invention, there is provided a peptide ligand specific for MT1-MMP comprising a polypeptide comprising at least three cysteine residues, separated by at least two loop sequences, and a molecular scaffold which forms covalent bonds with the cysteine residues of the polypeptide such that at least two polypeptide loops are formed on the molecular scaffold, characterised in that said molecular scaffold is 1,1′,1″-(1,3,5-triazinane-1,3,5-triyl)triprop-2-en-1-one (TATA).

According to a further aspect of the invention, there is provided a drug conjugate comprising a peptide ligand as defined herein conjugated to one or more effector and/or functional groups.

According to a further aspect of the invention, there is provided a pharmaceutical composition comprising a peptide ligand or a drug conjugate as defined herein in combination with one or more pharmaceutically acceptable excipients.

According to a further aspect of the invention, there is provided a peptide ligand or drug conjugate as defined herein for use in preventing, suppressing or treating a disease or disorder mediated by MT1-MMP.

### BRIEF DESCRIPTION OF THE FIGURES

FIG. 1: Body weight changes and Tumor volume trace after administering BT17BDC58 to female BALB/c nude mice bearing HT1080 xenograft. Data points represent group mean body weight. Error bars represent standard error of the mean (SEM).

### DETAILED DESCRIPTION OF THE INVENTION

In one embodiment, said loop sequences comprise 2, 3, 5, 6, 7 or 9 amino acids. In a further embodiment, said loop sequences comprise 3 or 7 amino acids.

In a further embodiment, said loop sequences comprise three cysteine residues separated by two loop sequences a first loop which consists of 7 amino acids and a second loop which consists of 2 amino acids, such as:

(SEQ ID NO: 1)
CEESFYPECDHC;

in particular:
A-(SEQ ID NO: 1)-A (herein referred to as 17-108-02).

In a further embodiment, said loop sequences comprise three cysteine residues separated by two loop sequences a first loop which consists of 3 amino acids and a second loop which consists of 6 amino acids, such as:

(SEQ ID NO: 2)
CPDLCLDLFPNC;
and
(SEQ ID NO: 3)
CPELCVDLYPHC;

in particular:
A-(SEQ ID NO: 2)-A (herein referred to as 17-111-01).
A-(SEQ ID NO: 3)-A (herein referred to as 17-111-02).

In a further embodiment, said loop sequences comprise three cysteine residues separated by two loop sequences a first loop which consists of 6 amino acids and a second loop which consists of 3 amino acids, such as:

(SEQ ID NO: 4)
CHPEWVSCEFHC;

in particular:
A-(SEQ ID NO: 4)-A (herein referred to as 17-116-01).

In a further embodiment, said loop sequences comprise three cysteine residues separated by two loop sequences a first loop which consists of 3 amino acids and a second loop which consists of 7 amino acids, such as

(SEQ ID NO: 5)
CSHECALLFPKTC;
(SEQ ID NO: 6)
CFDECQLLFPKTC;
(SEQ ID NO: 7)
CLDECKLLFPKTC;
(SEQ ID NO: 8)
CREECMLLFPKTC;
(SEQ ID NO: 9)
CETECALLFPRSC;
(SEQ ID NO: 10)
CADECRLLFPKTC;
(SEQ ID NO: 11)
CDVECRLLFPRSC;
(SEQ ID NO: 12)
CIDECRLLFPRSC;
(SEQ ID NO: 13)
CVRECALLFPKTC;
(SEQ ID NO: 14)
CV[HArg]ECALLFPKTC;
(SEQ ID NO: 15)
CVRECALLFPRTC;
(SEQ ID NO: 16)
CVRECALLFP[HArg]TC;
(SEQ ID NO: 17)
CV[HArg]ECALLFP[HArg]TC;
(SEQ ID NO: 18)
CV[HArg]ECALLFPATC;
(SEQ ID NO: 19)
CVAECALLFP[HArg]TC;
(SEQ ID NO: 20)
CVTECQLLFPKTC;
(SEQ ID NO: 21)
CRHECELLFPKTC;
(SEQ ID NO: 22)
CQRECALLFPKTC;
(SEQ ID NO: 23)
CVRECTLLFPKTC;
(SEQ ID NO: 24)
CTIECALLFPKTC;
(SEQ ID NO: 25)
CARECALLFPKTC;
(SEQ ID NO: 26)
CINECRLLFPKTC;
(SEQ ID NO: 27)
CYTECSLLFPKTC;
(SEQ ID NO: 28)
CHEECRLLFPKTC;
(SEQ ID NO: 29)
CLEECKLLFPKTC;
(SEQ ID NO: 30)
CIDECALLFPRTC;
(SEQ ID NO: 31)
CYEECRLLFPRTC;
(SEQ ID NO: 32)
CVRECRLLFPKTC;
(SEQ ID NO: 33)
CHIECALLFPKTC;
(SEQ ID NO: 34)
CKRECMLLFPKTC;
(SEQ ID NO: 35)
CYRECALLFPKTC;
(SEQ ID NO: 36)
CLTECALLFPKTC;
(SEQ ID NO: 37)
CEVECRLLFPKTC;
(SEQ ID NO: 38)
CEAECRLLFPKTC;
(SEQ ID NO: 39)
CVQECALLFPKTC;
(SEQ ID NO: 40)
CIRECSLLFPKTC;
(SEQ ID NO: 41)
CVTECALLFPKTC;
(SEQ ID NO: 42)
CVAECKLLFPKTC;
(SEQ ID NO: 43)
CVGECALLFPKTC;
(SEQ ID NO: 44)
CVVECALLFPKTC;
(SEQ ID NO: 45)
CVFECALLFPKTC;
(SEQ ID NO: 46)
CA[HArg]ECALLFP[HArg]TC;
(SEQ ID NO: 47)
CV[HArg]ECALLFA[HArg]TC;
(SEQ ID NO: 48)
CV[HArg]ECALLFP[HArg]AC;
(SEQ ID NO: 49)
CV[HArg]ECALL[1Nal]P[HArg]TC;
(SEQ ID NO: 50)
CV[HArg]ECALL[Cha]P[HArg]TC;
(SEQ ID NO: 51)
CV[HArg]ECALLF[Pip][HArg]TC;
(SEQ ID NO: 52)
CV[HArg]ECALLFP[HArg]SC;
(SEQ ID NO: 53)
CV[HArg]ECALLFP[HArg][HSer]C;
(SEQ ID NO: 54)
CV[HArg]ECALLF[HyP][HArg]TC;
(SEQ ID NO: 55)
CV[HArg]EC[Aib]LLFP[HArg]TC;
(SEQ ID NO: 56)
CV[HArg]ECAL[Nle]FP[HArg]TC;
(SEQ ID NO: 57)
CV[HArg]ECA[tBuAla]LFP[HArg]TC;
(SEQ ID NO: 58)
CV[HArg]ECA[Nle]LFP[HArg]TC;
(SEQ ID NO: 59)
CV[Aad2]ECALLFP[HArg]TC;
(SEQ ID NO: 60)
CP[HArg]ECALLFP[HArg]TC;
(SEQ NO: 61)
CV[HArg]ECALL[4FlPhe]P[HArg]TC;
(SEQ ID NO: 62)
CV[HArg]ECAL[tBuGly]FP[HArg]TC;
(SEQ ID NO: 63)
CV[HArg]ECAL[Cha]FP[HArg]TC;
(SEQ ID NO: 64)
CV[HArg]ECALL[2Nal]P[HArg]TC;
(SEQ ID NO: 65)
CV[HArg]ECALLFP[HArg][HyV]C;
(SEQ ID NO: 66)
C[tBuGly][HArg]ECALLFP[HArg]TC;
(SEQ ID NO: 67)
CVEECALLFP[HArg]TC;
(SEQ ID NO: 68)
CV[HArg]ECA[Cpa]LFP[HArg]TC;
(SEQ ID NO: 69)
CV[HArg]ECA[Cba]LFP[HArg]TC;
(SEQ ID NO: 70)
CV[HArg]ECA[C5A]LFP[HArg]TC;
(SEQ ID NO: 71)
CV[HArg]ECA[Cha]LFP[HArg]TC;
(SEQ ID NO: 72)
CV[HArg]ECA[tBuGly]LFP[HArg]TC;
(SEQ ID NO: 73)
CV[HArg]ECALLF[cis-HyP][HArg]TC;
(SEQ ID NO: 74)
CV[HArg]ECAL[Cpa]FP[HArg]TC;
(SEQ ID NO: 75)
CV[HArg]ECAL[C5A]FP[HArg]TC;
(SEQ ID NO: 76)
CV[HArg]ECA[tBuAla]LF[HyP][HArg]TC;
(SEQ ID NO: 77)
CV[HArg]ECA[tBuAla][tBuGly]F[Hylp][HArg]TC;
and
(SEQ ID NO: 78)
C[tBuGly][HArg]ECA[tBuAla]LFP[HArg]TC;

wherein Aad represents alpha-L-aminoadipic acid, Aib represents aminoisobutyric acid, C5a represents beta-cyclopentyl-L-alanine, Cba represents β-cyclobutylalanine, Cha represents 3-cyclohexyl-L-alanine, Cpa represents beta-cyclopropyl-L-alanine, 4FIPhe represents 4-fluoro-L-phenylalanine, HArg represents homoarginine, HyP represents hydroxyproline, HyV represents 3-hydroxy-L-valine, HSer represents homoserine, 1Nal represents 1-naphthylalanine, 2Nal represents 2-naphthylalanine, Ne represents norleucine, Pip represents pipecolic acid, tBuAla represents t-butyl-alanine, tBuGly represents t-butyl-glycine;

in particular:
A-(SEQ ID NO: 5)-A (herein referred to as 17-120-00);
A-(SEQ ID NO: 6)-A (herein referred to as 17-120-01);
A-(SEQ ID NO: 7)-A (herein referred to as 17-120-02);
A-(SEQ ID NO: 8)-A (herein referred to as 17-120-03);
A-(SEQ ID NO: 9)-A (herein referred to as 17-120-04);
A-(SEQ ID NO: 10)-A (herein referred to as 17-120-05);
A-(SEQ ID NO: 11)-A (herein referred to as 17-120-07);
A-(SEQ ID NO: 12)-A (herein referred to as 17-120-08);
APPP-(SEQ ID NO: 13)-A (herein referred to as 17-120-09-T01);
QISP-(SEQ ID NO: 13)-A (herein referred to as 17-120-09-T02);
ALPP-(SEQ ID NO: 13)-A (herein referred to as 17-120-09-T03 and BCY1124);
Ac-ALPP-(SEQ ID NO: 13) (herein referred to as Ac-(17-120-09-T03) and BCY1125);
Sar3-ALPP-(SEQ ID NO: 13) (herein referred to as Sar3-A-(17-120-09-T03));
GPPP-(SEQ ID NO: 13)-A (herein referred to as 17-120-09-T04);
SPPP-(SEQ ID NO: 13)-A (herein referred to as 17-120-09-T05);
NPPP-(SEQ ID NO: 13)-A (herein referred to as 17-120-09-T06);
EPPP-(SEQ ID NO: 13)-A (herein referred to as 17-120-09-T07);
HPPP-(SEQ ID NO: 13)-A (herein referred to as 17-120-09-T08);
APNP-(SEQ ID NO: 13)-A (herein referred to as 17-120-09-T09);
APDP-(SEQ ID NO: 13)-A (herein referred to as 17-120-09-T10);
APLP-(SEQ ID NO: 13)-A (herein referred to as 17-120-09-T11);
APAP-(SEQ ID NO: 13)-A (herein referred to as 17-120-09-T12);
APHP-(SEQ ID NO: 13)-A (herein referred to as 17-120-09-T13);
Sar3-ALPP-(SEQ ID NO: 14) (herein referred to as Sar3-A-(17-120-09-T03) HArg2);
Sar3-ALPP-(SEQ ID NO: 15) (herein referred to as Sar3-A-(17-120-09-T03) Arg9);
Sar3-ALPP-(SEQ ID NO: 16) (herein referred to as Sar3-A-(17-120-09-T03) HArg9);
(B-Ala)-Sar10-ALPP-(SEQ ID NO: 17) (herein referred to as (B-Ala)-Sar10-A-(17-120-09-T03) HArg2 HArg9);
Ac-(B-Ala)-Sar10-ALPP-(SEQ ID NO: 17) (herein referred to as Ac-(B-Ala)-Sar10-A-(17-120-09-T03) HArg2 HArg9);
ALPP-(SEQ ID NO: 17) (herein referred to as BCY3959);
[Ac]LPP-(SEQ ID NO: 17) (herein referred to as BCY9933);
[Ac]APP-(SEQ ID NO: 17) (herein referred to as BCY9934);
[Ac]LAP-(SEQ ID NO: 17) (herein referred to as BCY9935);
[Ac]LPA-(SEQ ID NO: 17) (herein referred to as BCY9936);
[Ac]-(SEQ ID NO: 17) (herein referred to as BCY9968);
[Ac]LYP-(SEQ ID NO: 17) (herein referred to as BCY11147);
[Ac]LPY-(SEQ ID NO: 17) (herein referred to as BCY11148);
[Ac][dA]PP-(SEQ ID NO: 17) (herein referred to as BCY11165);
[Ac]L[dA]P-(SEQ ID NO: 17) (herein referred to as BCY11166);
[Ac]LP[dA]-(SEQ ID NO: 17) (herein referred to as BCY11167);
ALPP-(SEQ ID NO: 17)-A (herein referred to as BCY10288);
(B-Ala)-Sar10-ALPP-(SEQ ID NO: 18) (herein referred to as (B-Ala)-Sar10-A-(17-120-09-T03) HArg2 Ala9);
(B-Ala)-Sar10-ALPP-(SEQ ID NO: 19) (herein referred to as (B-Ala)-Sar10-A-(17-120-09-T03) Ala2 HArg9);
[Ac]LPP-(SEQ ID NO: 19) (herein referred to as BCY9938);
APMP-(SEQ ID NO: 20)-A (herein referred to as 17-120-10-T01);
APSP-(SEQ ID NO: 21)-A (herein referred to as 17-120-11-T01);
AALP-(SEQ ID NO: 22)-A (herein referred to as 17-120-12-T01);
ALDP-(SEQ ID NO: 23)-A (herein referred to as 17-120-13-T01);
ADRP-(SEQ ID NO: 24)-A (herein referred to as 17-120-14-T01);
ATQP-(SEQ ID NO: 25)-A (herein referred to as 17-120-15-T01);
SPPP-(SEQ ID NO: 25)-A (herein referred to as 17-120-15-T02);
ARHP-(SEQ ID NO: 26)-A (herein referred to as 17-120-16-T01);
ALPP-(SEQ ID NO: 27)-A (herein referred to as 17-120-17-T01);
A-(SEQ ID NO: 28)-A (herein referred to as 17-120-18);
A-(SEQ ID NO: 29)-A (herein referred to as 17-120-19);
A-(SEQ ID NO: 30)-A (herein referred to as 17-120-20);
A-(SEQ ID NO: 31)-A (herein referred to as 17-120-21);
APPP-(SEQ ID NO: 31)-A (herein referred to as 17-120-21-T01);
APSP-(SEQ ID NO: 32)-A (herein referred to as 17-120-22-T01);
PLPP-(SEQ ID NO: 32)-A (herein referred to as 17-120-22-T02);
APAP-(SEQ ID NO: 33)-A (herein referred to as 17-120-23-T01);
AVEP-(SEQ ID NO: 34)-A (herein referred to as 17-120-24-T01);
AEPA-(SEQ ID NO: 35)-A (herein referred to as 17-120-25-T01);
ASPP-(SEQ ID NO: 36)-A (herein referred to as 17-120-26-T01);
AAPP-(SEQ ID NO: 37)-A (herein referred to as 17-120-27-T01);
APPP-(SEQ ID NO: 38)-A (herein referred to as 17-120-28-T01);
AVPP-(SEQ ID NO: 39)-A (herein referred to as 17-120-29-T01);
SPPP-(SEQ ID NO: 40)-A (herein referred to as 17-120-30-T01);
HLPP-(SEQ ID NO: 41)-A (herein referred to as 17-120-31-T01);
RLPP-(SEQ ID NO: 41)-A (herein referred to as 17-120-31-T02);
APPP-(SEQ ID NO: 41)-A (herein referred to as 17-120-31-T03);
MPPP-(SEQ ID NO: 42)-A (herein referred to as 17-120-32-T01);
SPPP-(SEQ ID NO: 43)-A (herein referred to as 17-120-33-T01);
APPP-(SEQ ID NO: 44)-A (herein referred to as 17-120-34-T01);
APPP-(SEQ ID NO: 45)-A (herein referred to as 17-120-35-T01);
[Ac]LPP-(SEQ ID NO: 46) (herein referred to as BCY9937);
[Ac]LPP-(SEQ ID NO: 47) (herein referred to as BCY9943);
[Ac]LPP-(SEQ ID NO: 48) (herein referred to as BCY9945);
[Ac]LPP-(SEQ ID NO: 49) (herein referred to as BCY9946);
[Ac]LPP-(SEQ ID NO: 50) (herein referred to as BCY9949);
[Ac]LPP-(SEQ ID NO: 51) (herein referred to as BCY9951);
[Ac]LPP-(SEQ ID NO: 52) (herein referred to as BCY9952);
[Ac]LPP-(SEQ ID NO: 53) (herein referred to as BCY9953);
[Ac]LPP-(SEQ ID NO: 54) (herein referred to as BCY9954);
[Ac]LPP-(SEQ ID NO: 55) (herein referred to as BCY9955);
[Ac]LPP-(SEQ ID NO: 56) (herein referred to as BCY9957);
[Ac]LPP-(SEQ ID NO: 57) (herein referred to as BCY9959);
[Ac]LYP-(SEQ ID NO: 57) (herein referred to as BCY12401);
[Ac]EYP-(SEQ ID NO: 57) (herein referred to as BCY12405);
[Ac]LPP-(SEQ ID NO: 58) (herein referred to as BCY9960);
[Ac]LPP-(SEQ ID NO: 59) (herein referred to as BCY9961);
[Ac]LPP-(SEQ ID NO: 60) (herein referred to as BCY9963);
[Ac]LPP-(SEQ ID NO: 61) (herein referred to as BCY9964);
[Ac]LPP-(SEQ ID NO: 62) (herein referred to as BCY9965);
[Ac]LPP-(SEQ ID NO: 63) (herein referred to as BCY9966);
[Ac]LPP-(SEQ ID NO: 64) (herein referred to as BCY10223);
[Ac]LPP-(SEQ ID NO: 65) (herein referred to as BCY10224);
[Ac]LPP-(SEQ ID NO: 66) (herein referred to as BCY11149);
[Ac]LPP-(SEQ ID NO: 67) (herein referred to as BCY11150);
[Ac]LPP-(SEQ ID NO: 68) (herein referred to as BCY11151);
[Ac]LPP-(SEQ ID NO: 69) (herein referred to as BCY11152);
[Ac]LPP-(SEQ ID NO: 70) (herein referred to as BCY11153);
[Ac]LPP-(SEQ ID NO: 71) (herein referred to as BCY11154);
[Ac]LPP-(SEQ ID NO: 72) (herein referred to as BCY11155);
[Ac]LPP-(SEQ ID NO: 73) (herein referred to as BCY11163);
[Ac]LPP-(SEQ ID NO: 74) (herein referred to as BCY11158);
[Ac]LPP-(SEQ ID NO: 75) (herein referred to as BCY11160);
[Ac]LYP-(SEQ ID NO: 76) (herein referred to as BCY12402);
[Ac]LYP-(SEQ ID NO: 77) (herein referred to as BCY12403); and
[Ac]LYP-(SEQ ID NO: 78) (herein referred to as BCY12404).

In a further embodiment, the peptide ligand comprises an amino acid sequence which is (B-Ala)-Sar10-ALPP-(SEQ ID NO: 17) (herein referred to as (B-Ala)-Sar10-A-(17-120-09-T03) HArg2 HArg9).

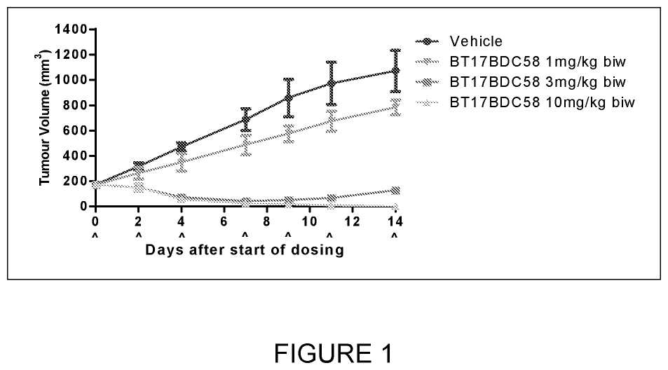

Data is presented herein in FIG. 1 and Tables 4 and 5 that a bicyclic peptide drug conjugate containing this peptide ligand (BT17BDC58) produced dose-dependent antitumor activity.

In a further embodiment, said loop sequences comprise three cysteine residues separated by two loop sequences a first loop which consists of 7 amino acids and a second loop which consists of 3 amino acids, such as:

(SEQ ID NO: 79)
CSSWDKLMCHPYC;

in particular:
A-(SEQ ID NO: 79)-A (herein referred to as 17-121-00).

In a further embodiment, said loop sequences comprise three cysteine residues separated by two loop sequences a first loop which consists of 3 amino acids and a second loop which consists of 9 amino acids, such as:

(SEQ ID NO: 80)
CPEECFYLPPHPMSC;
(SEQ ID NO: 81)
CPQECFYLPGHSLYC;
(SEQ ID NO: 82)
CPGECFYPPGHPLAC;
(SEQ ID NO: 83)
CPGECFYPTNHPLYC;
(SEQ ID NO: 84)
CPQECFYPIGHPLAC;
(SEQ ID NO: 85)
CPEECFYPPGHKLHC;
(SEQ ID NO: 86)
CPQECFYPPGHRLRC;
(SEQ ID NO: 87)
CPQECFYPPGHPYHC;
(SEQ ID NO: 88)
CPQECFYPSTHPLYC;
(SEQ ID NO: 89)
CPGECFYPSNHRLYC;
(SEQ ID NO: 90)
CPDECFYPPEHPLAC;
(SEQ ID NO: 91)
CPGECFYPPGHHLSC;
(SEQ ID NO: 92)
CPGECFYPPGHHLGC;
(SEQ ID NO: 93)
CPEECFYPPNHPLYC;
(SEQ ID NO: 94)
CPGECFYPPDHPLYC;
(SEQ ID NO: 95)
CPGECFYPPGHPLYC;
(SEQ ID NO: 96)
CPGECFYPPNHPFYC;
(SEQ ID NO: 97)
CPGECFYPPNHPLYC;
(SEQ ID NO: 98)
CPEECFYPPGHPLAC;
(SEQ ID NO: 99)
CWMECFYPPGHPLAC;
(SEQ ID NO: 100)
CFEECFYPPGHPLAC;
(SEQ ID NO: 101)
CPGECFYPPGHPLRC;
(SEQ ID NO: 102)
CPGECFYPPGHPREC;
(SEQ ID NO: 103)
CPGECFYPPGHRFHC;
and
(SEQ ID NO: 104)
CPGECFYPPGHRLYC;

in particular:
A-(SEQ ID NO: 80)-A (herein referred to as 17-127-01);
A-(SEQ ID NO: 81)-A (herein referred to as 17-129-00);
SQT-(SEQ ID NO: 82)-A (herein referred to as 17-129-01-T01);
SMT-(SEQ ID NO: 82)-A (herein referred to as 17-129-01-T02);
SLV-(SEQ ID NO: 82)-A (herein referred to as 17-129-01-T03);
ISSYG-(SEQ ID NO: 82)-A (herein referred to as 17-129-01-T04);
ENITT-(SEQ ID NO: 82)-A (herein referred to as 17-129-01-T05);
A-(SEQ ID NO: 83)-A (herein referred to as 17-129-02);
A-(SEQ ID NO: 84)-A (herein referred to as 17-129-03);
A-(SEQ ID NO: 85)-A (herein referred to as 17-129-04);
A-(SEQ ID NO: 86)-A (herein referred to as 17-129-05);
A-(SEQ ID NO: 87)-A (herein referred to as 17-129-06);
A-(SEQ ID NO: 88)-A (herein referred to as 17-129-07);
A-(SEQ ID NO: 89)-A (herein referred to as 17-129-08);
A-(SEQ ID NO: 90)-A (herein referred to as 17-129-09);
A-(SEQ ID NO: 91)-A (herein referred to as 17-129-10);
A-(SEQ ID NO: 92)-A (herein referred to as 17-129-11);
L-(SEQ ID NO: 93)-HA (herein referred to as 17-129-12-T01);
T-(SEQ ID NO: 94)-NA (herein referred to as 17-129-13-T01);
Q-(SEQ ID NO: 95)-NA (herein referred to as 17-129-14-T01);
A-(SEQ ID NO: 95)-NVI (herein referred to as 17-129-14-T02);
N-(SEQ ID NO: 96)-NA (herein referred to as 17-129-15-T01);
D-(SEQ ID NO: 97)-RA (herein referred to as 17-129-16-T01);
SRM-(SEQ ID NO: 98)-A (herein referred to as 17-129-17-T01);
SRS-(SEQ ID NO: 98)-A (herein referred to as 17-129-17-T02);
RYMTR-(SEQ ID NO: 98)-A (herein referred to as 17-129-17-T03);
REE-(SEQ ID NO: 99)-A (herein referred to as 17-129-18-T01);
DNM-(SEQ ID NO: 99)-A (herein referred to as 17-129-18-T02);
QES-(SEQ ID NO: 99)-A (herein referred to as 17-129-18-T03);
ADY-(SEQ ID NO: 99)-A (herein referred to as 17-129-18-T04);
MAN-(SEQ ID NO: 100)-A (herein referred to as 17-129-19-T01);
SQN-(SEQ ID NO: 100)-A (herein referred to as 17-129-19-T02);
A-(SEQ ID NO: 101)-TVL (herein referred to as 17-129-20-T01);
A-(SEQ ID NO: 102)-SWL (herein referred to as 17-129-21-T01);
A-(SEQ ID NO: 103)-LTE (herein referred to as 17-129-22-T01);
A-(SEQ ID NO: 104)-YSE (herein referred to as 17-129-23-T01); and
Ac-(SEQ ID NO: 104)-YSE (herein referred to as Ac(17-129-23-T01)).

In a further embodiment, said loop sequences comprise three cysteine residues separated by two loop sequences a first loop which consists of 6 amino acids and a second loop which consists of 6 amino acids, such as

(SEQ ID NO: 105)
CEEEFYPCGHPLYVC;
(SEQ ID NO: 106)
CEEQFYPCTHALYTC;
(SEQ ID NO: 107)
CVEEFYPCDHPLYSC;
(SEQ ID NO: 108)
CEEEFYPCGHPMHPC;
(SEQ ID NO: 109)
CDEQFYPCHHRLYSC;
(SEQ ID NO: 110)
CEEEFYPCGHPFHPC;
(SEQ ID NO: 111)
CLEQFYPCEHPLFSC;
(SEQ ID NO: 112)
CVEQFYPCGHRHYIC;
(SEQ ID NO: 113)
CEEQFYPCSHPLYTC;
(SEQ ID NO: 114)
CEEQFYPCNHPLNVC;
(SEQ ID NO: 115)
CEEEFYPCSHPLNPC;
(SEQ ID NO: 116)
CEEQFYPCGHKLSPC;
(SEQ ID NO: 117)
CPEQFYPCDHRLYIC;
(SEQ ID NO: 118)
CQEQFYPCNHPLSPC;
(SEQ ID NO: 119)
CDEQFYPCNHRLNTC;
(SEQ ID NO: 120)
CEEAFYPCHHPLYRC;
(SEQ ID NO: 121)
CDEDFYPCGHYLNQC;
(SEQ ID NO: 122)
CEEQFYPCTHPLYVC;
(SEQ ID NO: 123)
CPEQFYPCTHRLYQC;
(SEQ ID NO: 124)
CEEQFYPCSHPLYRC;
(SEQ ID NO: 125)
CAEQFYPCDHPLYRC;
(SEQ ID NO: 126)
CAEEFYPCDHPLYRC;
(SEQ ID NO: 127)
CEEAFYPCNHPLYTC;
(SEQ ID NO: 128)
CAEAFYPCDHPLYVC;
(SEQ ID NO: 129)
CEEAFYPCSHPLFIC;
(SEQ ID NO: 130)
CEEAFYPCSHPLHPC;
(SEQ ID NO: 131)
CEEAFYPCSHPLFVC;
(SEQ ID NO: 132)
CEEQFYPCSHPLYSC;
(SEQ ID NO: 133)
CEEAFYPCEHPLYMC;
and
(SEQ ID NO: 134)
CEEQFYPCNHPLYMC;

in particular:
A-(SEQ ID NO: 105)-A (herein referred to as 17-126-01);
A-(SEQ ID NO: 106)-A (herein referred to as 17-126-02);
A-(SEQ ID NO: 107)-A (herein referred to as 17-126-03);
A-(SEQ ID NO: 108)-A (herein referred to as 17-126-06);
A-(SEQ ID NO: 109)-A (herein referred to as 17-126-07);
A-(SEQ ID NO: 110)-A (herein referred to as 17-126-08);
A-(SEQ ID NO: 111)-A (herein referred to as 17-126-09);
A-(SEQ ID NO: 112)-A (herein referred to as 17-126-10);
A-(SEQ ID NO: 113)-A (herein referred to as 17-126-18);
A-(SEQ ID NO: 114)-A (herein referred to as 17-126-19);
A-(SEQ ID NO: 115)-A (herein referred to as 17-126-20);
A-(SEQ ID NO: 116)-A (herein referred to as 17-126-21);
A-(SEQ ID NO: 117)-A (herein referred to as 17-126-22);
A-(SEQ ID NO: 118)-A (herein referred to as 17-126-23);
A-(SEQ ID NO: 119)-A (herein referred to as 17-126-24);
A-(SEQ ID NO: 120)-A (herein referred to as 17-126-25);
Ac-A-(SEQ ID NO: 120)-A (herein referred to as Ac-(17-126-25));
A-(SEQ ID NO: 121)-A (herein referred to as 17-126-26);
A-(SEQ ID NO: 122)-A (herein referred to as 17-126-27);
A-(SEQ ID NO: 123)-A (herein referred to as 17-126-28);
HSP-(SEQ ID NO: 124)-A (herein referred to as 17-126-30-T01);
GPH-(SEQ ID NO: 125)-A (herein referred to as 17-126-31-T01);
IHS-(SEQ ID NO: 126)-A (herein referred to as 17-126-32-T01);
WSP-(SEQ ID NO: 127)-A (herein referred to as 17-126-33-T01);
SHS-(SEQ ID NO: 127)-A (herein referred to as 17-126-33-T02);
DLH-(SEQ ID NO: 128)-A (herein referred to as 17-126-35-T01);
ANE-(SEQ ID NO: 129)-A (herein referred to as 17-126-36-T01);
AVW-(SEQ ID NO: 130)-A (herein referred to as 17-126-37-T01);
KVQ-(SEQ ID NO: 131)-A (herein referred to as 17-126-38-T01);
A-(SEQ ID NO: 132)-PDVA (herein referred to as 17-126-39-T01);
A-(SEQ ID NO: 133)-HQAA (herein referred to as 17-126-40-T01); and
A-(SEQ ID NO: 134)-RENA (herein referred to as 17-126-41-T01).

In a further embodiment, said loop sequences comprise three cysteine residues separated by two loop sequences a first loop which consists of 6 amino acids and a second loop which consists of 5 amino acids, such as:

(SEQ ID NO: 135)
CLEQFYPCGDPRLC;
and
(SEQ ID NO: 136)
CEEQFYPCGHHLLC;

in particular:
A-(SEQ ID NO: 135)-A (herein referred to as 17-126-11); and
A-(SEQ ID NO: 136)-A (herein referred to as 17-126-12).

In a further embodiment, said loop sequences comprise three cysteine residues separated by two loop sequences a first loop which consists of 5 amino acids and a second loop which consists of 5 amino acids, such as:

(SEQ ID NO: 137)
CLEPDECFYPMEC;
(SEQ ID NO: 138)
CKEPQECFYPLKC;
and
(SEQ ID NO: 139)
CDSPEECFYPLEC;

in particular:
A-(SEQ ID NO: 137)-A (herein referred to as 17-122-02);
A-(SEQ ID NO: 138)-A (herein referred to as 17-122-03); and
A-(SEQ ID NO: 139)-A (herein referred to as 17-122-04).

In one embodiment, the peptide ligand is selected from any of the peptide ligands listed in Table 2 or Table 3.

Unless defined otherwise, all technical and scientific terms used herein have the same meaning as commonly understood by those of ordinary skill in the art, such as in the arts of peptide chemistry, cell culture and phage display, nucleic acid chemistry and biochemistry.

Standard techniques are used for molecular biology, genetic and biochemical methods (see Sambrook et al., Molecular Cloning: A Laboratory Manual, 3rd ed., 2001, Cold Spring Harbor Laboratory Press, Cold Spring Harbor, N.Y.; Ausubel et al., Short Protocols in Molecular Biology (1999) 4^{th} ed., John Wiley & Sons, Inc.), which are incorporated herein by reference.

### Nomenclature

Numbering

When referring to amino acid residue positions within peptide ligands of the invention, cysteine residues (Cᵢ, Cᵢᵢ and Cᵢᵢᵢ) are omitted from the numbering as they are invariant, therefore, the numbering of amino acid residues within peptide ligands of the invention is referred to as below:

(SEQ ID NO: 1)
Cᵢ-E₁-E₂-S₃-F₄-Y₅-P₆-E₇-Cᵢᵢ-D₈-H₉-Cᵢᵢᵢ.

For the purpose of this description, all bicyclic peptides are assumed to be cyclised with 1,1′,1″-(1,3,5-triazinane-1,3,5-triyl)tripropan-1-one (TATA) and yielding a tri-substituted structure. Cyclisation with TATA occurs on Cᵢ, Cᵢᵢ, and Cᵢᵢᵢ. TATA is an example of an αβ unsaturated carbonyl containing molecular scaffold (Angewandte Chemie, International Edition (2014), 53(6), 1602-1606).

Molecular Format

N- or C-terminal extensions to the bicycle core sequence are added to the left or right side of the sequence, separated by a hyphen. For example, an N-terminal βAla-Sar10-Ala tail would be denoted as:

(SEQ ID NO: X)
βAla-Sar10-A.

Inversed Peptide Sequences

In light of the disclosure in Nair et al (2003) J Immunol 170(3), 1362-1373, it is envisaged that the peptide sequences disclosed herein would also find utility in their retro-inverso form. For example, the sequence is reversed (i.e. N-terminus becomes C-terminus and vice versa) and their stereochemistry is likewise also reversed (i.e. D-amino acids become L-amino acids and vice versa).

Peptide Ligands

A peptide ligand, as referred to herein, refers to a peptide covalently bound to a molecular scaffold. Typically, such peptides comprise two or more reactive groups (i.e. cysteine residues) which are capable of forming covalent bonds to the scaffold, and a sequence subtended between said reactive groups which is referred to as the loop sequence, since it forms a loop when the peptide is bound to the scaffold. In the present case, the peptides comprise at least three cysteine residues (referred to herein as Cᵢ, Cᵢᵢ and Cᵢᵢᵢ), and form at least two loops on the scaffold.

Advantages of the Peptide Ligands

Certain bicyclic peptides of the present invention have a number of advantageous properties which enable them to be considered as suitable drug-like molecules for injection, inhalation, nasal, ocular, oral or topical administration. Such advantageous properties include:
Species cross-reactivity. Certain ligands demonstrate cross-reactivity across PBPs from different bacterial species and hence are able to treat infections caused by multiple species of bacteria. Other ligands may be highly specific for the PBPs of certain bacterial species which may be advantageous for treating an infection without collateral damage to the beneficial flora of the patient;
Protease stability. Bicyclic peptide ligands should ideally demonstrate stability to plasma proteases, epithelial (“membrane-anchored”) proteases, gastric and intestinal proteases, lung surface proteases, intracellular proteases and the like. Protease stability should be maintained between different species such that a bicycle lead candidate can be developed in animal models as well as administered with confidence to humans;
Desirable solubility profile. This is a function of the proportion of charged and hydrophilic versus hydrophobic residues and intra/inter-molecular H-bonding, which is important for formulation and absorption purposes;
An optimal plasma half-life in the circulation. Depending upon the clinical indication and treatment regimen, it may be required to develop a bicyclic peptide for short exposure in an acute illness management setting, or develop a bicyclic peptide with enhanced retention in the circulation, and is therefore optimal for the management of more chronic disease states. Other factors driving the desirable plasma half-life are requirements of sustained exposure for maximal therapeutic efficiency versus the accompanying toxicology due to sustained exposure of the agent; and
Selectivity. Certain peptide ligands of the invention demonstrate selectivity for MT1-MMP, but does not cross-react with MMP isoforms, such as MMP-1, MMP-2, MMP-15 and MMP-16.

Pharmaceutically Acceptable Salts

It will be appreciated that salt forms are within the scope of this invention, and references to peptide ligands include the salt forms of said ligands.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two.

Acid addition salts (mono- or di-salts) may be formed with a wide variety of acids, both inorganic and organic. Examples of acid addition salts include mono- or di-salts formed with an acid selected from the group consisting of acetic, 2,2-dichloroacetic, adipic, alginic, ascorbic (e.g. L-ascorbic), L-aspartic, benzenesulfonic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrohalic acids (e.g. hydrobromic, hydrochloric, hydriodic), isethionic, lactic (e.g. (+)-L-lactic, (±)-DL-lactic), lactobionic, maleic, malic, (−)-L-malic, malonic, (±)-DL-mandelic, methanesulfonic, naphthalene-2-sulfonic, naphthalene-1,5-disulfonic, 1-hydroxy-2-naphthoic, nicotinic, nitric, oleic, orotic, oxalic, palmitic, pamoic, phosphoric, propionic, pyruvic, L-pyroglutamic, salicylic, 4-amino-salicylic, sebacic, stearic, succinic, sulfuric, tannic, (+)-L-tartaric, thiocyanic, p-toluenesulfonic, undecylenic and valeric acids, as well as acylated amino acids and cation exchange resins.

One particular group of salts consists of salts formed from acetic, hydrochloric, hydriodic, phosphoric, nitric, sulfuric, citric, lactic, succinic, maleic, malic, isethionic, fumaric, benzenesulfonic, toluenesulfonic, sulfuric, methanesulfonic (mesylate), ethanesulfonic, naphthalenesulfonic, valeric, propanoic, butanoic, malonic, glucuronic and lactobionic acids. One particular salt is the hydrochloride salt. Another particular salt is the acetate salt.

If the compound is anionic, or has a functional group which may be anionic (e.g., —COOH may be —COO⁻), then a salt may be formed with an organic or inorganic base, generating a suitable cation. Examples of suitable inorganic cations include, but are not limited to, alkali metal ions such as Li⁺, Na⁺ and K⁺, alkaline earth metal cations such as Ca²⁺ and Mg²⁺, and other cations such as Al³⁺ or Zn⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: methylamine, ethylamine, diethylamine, propylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the peptides of the invention contain an amine function, these may form quaternary ammonium salts, for example by reaction with an alkylating agent according to methods well known to the skilled person. Such quaternary ammonium compounds are within the scope of the peptides of the invention.

Modified Derivatives

It will be appreciated that modified derivatives of the peptide ligands as defined herein are within the scope of the present invention. Examples of such suitable modified derivatives include one or more modifications selected from: N-terminal and/or C-terminal modifications; replacement of one or more amino acid residues with one or more non-natural amino acid residues (such as replacement of one or more polar amino acid residues with one or more isosteric or isoelectronic amino acids; replacement of one or more non-polar amino acid residues with other non-natural isosteric or isoelectronic amino acids); addition of a spacer group; replacement of one or more oxidation sensitive amino acid residues with one or more oxidation resistant amino acid residues; replacement of one or more amino acid residues with an alanine, replacement of one or more L-amino acid residues with one or more D-amino acid residues; N-alkylation of one or more amide bonds within the bicyclic peptide ligand; replacement of one or more peptide bonds with a surrogate bond; peptide backbone length modification; substitution of the hydrogen on the alpha-carbon of one or more amino acid residues with another chemical group, modification of amino acids such as cysteine, lysine, glutamate/aspartate and tyrosine with suitable amine, thiol, carboxylic acid and phenol-reactive reagents so as to functionalise said amino acids, and introduction or replacement of amino acids that introduce orthogonal reactivities that are suitable for functionalisation, for example azide or alkyne-group bearing amino acids that allow functionalisation with alkyne or azide-bearing moieties, respectively.

In one embodiment, the modified derivative comprises an N-terminal and/or C-terminal modification. In a further embodiment, wherein the modified derivative comprises an N-terminal modification using suitable amino-reactive chemistry, and/or C-terminal modification using suitable carboxy-reactive chemistry. In a further embodiment, said N-terminal or C-terminal modification comprises addition of an effector group, including but not limited to a cytotoxic agent, a radiochelator or a chromophore.

In a further embodiment, the modified derivative comprises an N-terminal modification. In a further embodiment, the N-terminal modification comprises an N-terminal acetyl group. In this embodiment, the N-terminal cysteine group (the group referred to herein as Cᵢ) is capped with acetic anhydride or other appropriate reagents during peptide synthesis leading to a molecule which is N-terminally acetylated. This embodiment provides the advantage of removing a potential recognition point for aminopeptidases and avoids the potential for degradation of the bicyclic peptide.

In an alternative embodiment, the N-terminal modification comprises the addition of a molecular spacer group which facilitates the conjugation of effector groups and retention of potency of the bicyclic peptide to its target.

In a further embodiment, the modified derivative comprises a C-terminal modification. In a further embodiment, the C-terminal modification comprises an amide group. In this embodiment, the C-terminal cysteine group (the group referred to herein as Cᵢᵢᵢ) is synthesized as an amide during peptide synthesis leading to a molecule which is C-terminally amidated. This embodiment provides the advantage of removing a potential recognition point for carboxypeptidase and reduces the potential for proteolytic degradation of the bicyclic peptide.

In one embodiment, the modified derivative comprises replacement of one or more amino acid residues with one or more non-natural amino acid residues. In this embodiment, non-natural amino acids may be selected having isosteric/isoelectronic side chains which are neither recognised by degradative proteases nor have any adverse effect upon target potency.

Alternatively, non-natural amino acids may be used having constrained amino acid side chains, such that proteolytic hydrolysis of the nearby peptide bond is conformationally and sterically impeded. In particular, these concern proline analogues, bulky sidechains, CD-disubstituted derivatives (for example, aminoisobutyric acid, Aib), and cyclo amino acids, a simple derivative being amino-cyclopropylcarboxylic acid.

In one embodiment, the modified derivative comprises the addition of a spacer group. In a further embodiment, the modified derivative comprises the addition of a spacer group to the N-terminal cysteine (Cᵢ) and/or the C-terminal cysteine (Cᵢᵢᵢ).

In one embodiment, the modified derivative comprises replacement of one or more oxidation sensitive amino acid residues with one or more oxidation resistant amino acid residues.

In one embodiment, the modified derivative comprises replacement of one or more charged amino acid residues with one or more hydrophobic amino acid residues. In an alternative embodiment, the modified derivative comprises replacement of one or more hydrophobic amino acid residues with one or more charged amino acid residues. The correct balance of charged versus hydrophobic amino acid residues is an important characteristic of the bicyclic peptide ligands. For example, hydrophobic amino acid residues influence the degree of plasma protein binding and thus the concentration of the free available fraction in plasma, while charged amino acid residues (in particular arginine) may influence the interaction of the peptide with the phospholipid membranes on cell surfaces. The two in combination may influence half-life, volume of distribution and exposure of the peptide drug, and can be tailored according to the clinical endpoint. In addition, the correct combination and number of charged versus hydrophobic amino acid residues may reduce irritation at the injection site (if the peptide drug has been administered subcutaneously).

In one embodiment, the modified derivative comprises replacement of one or more L-amino acid residues with one or more D-amino acid residues. This embodiment is believed to increase proteolytic stability by steric hindrance and by a propensity of D-amino acids to stabilise β-turn conformations (Tugyi et al (2005) PNAS, 102(2), 413-418).

In one embodiment, the modified derivative comprises removal of any amino acid residues and substitution with alanines. This embodiment provides the advantage of removing potential proteolytic attack site(s).

It should be noted that each of the above mentioned modifications serve to deliberately improve the potency or stability of the peptide. Further potency improvements based on modifications may be achieved through the following mechanisms:
Incorporating hydrophobic moieties that exploit the hydrophobic effect and lead to lower off rates, such that higher affinities are achieved;
Incorporating charged groups that exploit long-range ionic interactions, leading to faster on rates and to higher affinities (see for example Schreiber et al, Rapid, electrostatically assisted association of proteins (1996), Nature Struct. Biol. 3, 427-31); and
Incorporating additional constraint into the peptide, by for example constraining side chains of amino acids correctly such that loss in entropy is minimal upon target binding, constraining the torsional angles of the backbone such that loss in entropy is minimal upon target binding and introducing additional cyclisations in the molecule for identical reasons. (for reviews see Gentilucci et al, Curr. Pharmaceutical Design, (2010), 16, 3185-203, and Nestor et al, Curr. Medicinal Chem (2009), 16, 4399-418).

Isotopic Variations

The present invention includes all pharmaceutically acceptable (radio)isotope-labeled peptide ligands of the invention, wherein one or more atoms are replaced by atoms having the same atomic number, but an atomic mass or mass number different from the atomic mass or mass number usually found in nature, and peptide ligands of the invention, wherein metal chelating groups are attached (termed “effector”) that are capable of holding relevant (radio)isotopes, and peptide ligands of the invention, wherein certain functional groups are covalently replaced with relevant (radio)isotopes or isotopically labelled functional groups.

Examples of isotopes suitable for inclusion in the peptide ligands of the invention comprise isotopes of hydrogen, such as ²H (D) and ³H (T), carbon, such as ¹¹C, ¹³C and ¹⁴C, chlorine, such as ³⁶Cl, fluorine, such as ¹⁸F, iodine, such as ¹²³I, ¹²⁵I and ¹³¹I, nitrogen, such as ¹³N and ¹⁵N, oxygen, such as ¹⁵O, ¹⁷O and ¹⁸O, phosphorus, such as ³²P, sulfur, such as ³⁵S, copper, such as ⁶⁴Cu, gallium, such as ⁶⁷Ga or ⁶⁸Ga, yttrium, such as ⁹⁰Y and lutetium, such as ¹⁷⁷Lu, and Bismuth, such as ²¹³Bi.

Certain isotopically-labelled peptide ligands of the invention, for example, those incorporating a radioactive isotope, are useful in drug and/or substrate tissue distribution studies. The peptide ligands of the invention can further have valuable diagnostic properties in that they can be used for detecting or identifying the formation of a complex between a labelled compound and other molecules, peptides, proteins, enzymes or receptors. The detecting or identifying methods can use compounds that are labelled with labelling agents such as radioisotopes, enzymes, fluorescent substances, luminous substances (for example, luminol, luminol derivatives, luciferin, aequorin and luciferase), etc. The radioactive isotopes tritium, i.e. ³H (T), and carbon-14, i.e. ¹⁴C, are particularly useful for this purpose in view of their ease of incorporation and ready means of detection.

Substitution with heavier isotopes such as deuterium, i.e. ²H (D), may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining target occupancy.

Isotopically-labeled compounds of peptide ligands of the invention can generally be prepared by conventional techniques known to those skilled in the art or by processes analogous to those described in the accompanying Examples using an appropriate isotopically-labeled reagent in place of the non-labeled reagent previously employed.

Effector and Functional Groups

According to a further aspect of the invention, there is provided a drug conjugate comprising a peptide ligand as defined herein conjugated to one or more effector and/or functional groups.

Effector and/or functional groups can be attached, for example, to the N and/or C termini of the polypeptide, to an amino acid within the polypeptide, or to the molecular scaffold.

Appropriate effector groups include antibodies and parts or fragments thereof. For instance, an effector group can include an antibody light chain constant region (CL), an antibody CH1 heavy chain domain, an antibody CH2 heavy chain domain, an antibody CH3 heavy chain domain, or any combination thereof, in addition to the one or more constant region domains. An effector group may also comprise a hinge region of an antibody (such a region normally being found between the CH1 and CH2 domains of an IgG molecule).

In a further embodiment of this aspect of the invention, an effector group according to the present invention is an Fc region of an IgG molecule. Advantageously, a peptide ligand-effector group according to the present invention comprises or consists of a peptide ligand Fc fusion having a tβ half-life of a day or more, two days or more, 3 days or more, 4 days or more, 5 days or more, 6 days or more or 7 days or more. Most advantageously, the peptide ligand according to the present invention comprises or consists of a peptide ligand Fc fusion having a tβ half-life of a day or more.

Functional groups include, in general, binding groups, drugs, reactive groups for the attachment of other entities, functional groups which aid uptake of the macrocyclic peptides into cells, and the like.

The ability of peptides to penetrate into cells will allow peptides against intracellular targets to be effective. Targets that can be accessed by peptides with the ability to penetrate into cells include transcription factors, intracellular signalling molecules such as tyrosine kinases and molecules involved in the apoptotic pathway. Functional groups which enable the penetration of cells include peptides or chemical groups which have been added either to the peptide or the molecular scaffold. Peptides such as those derived from such as VP22, HIV-Tat, a homeobox protein of Drosophila (Antennapedia), e.g. as described in Chen and Harrison, Biochemical Society Transactions (2007) Volume 35, part 4, p 821; Gupta et al. in Advanced Drug Discovery Reviews (2004) Volume 57 9637. Examples of short peptides which have been shown to be efficient at translocation through plasma membranes include the 16 amino acid penetratin peptide from Drosophila Antennapedia protein (Derossi et al (1994) J Biol. Chem. Volume 269 p 10444), the 18 amino acid ‘model amphipathic peptide’ (Oehlke et al (1998) Biochim Biophys Acts Volume 1414 p 127) and arginine rich regions of the HIV TAT protein. Non peptidic approaches include the use of small molecule mimics or SMOCs that can be easily attached to biomolecules (Okuyama et al (2007) Nature Methods Volume 4 p 153). Other chemical strategies to add guanidinium groups to molecules also enhance cell penetration (Elson-Scwab et al (2007) J Biol Chem Volume 282 p 13585). Small molecular weight molecules such as steroids may be added to the molecular scaffold to enhance uptake into cells.

One class of functional groups which may be attached to peptide ligands includes antibodies and binding fragments thereof, such as Fab, Fv or single domain fragments. In particular, antibodies which bind to proteins capable of increasing the half-life of the peptide ligand in vivo may be used.

In one embodiment, a peptide ligand-effector group according to the invention has a tβ half-life selected from the group consisting of: 12 hours or more, 24 hours or more, 2 days or more, 3 days or more, 4 days or more, 5 days or more, 6 days or more, 7 days or more, 8 days or more, 9 days or more, 10 days or more, 11 days or more, 12 days or more, 13 days or more, 14 days or more, 15 days or more or 20 days or more. Advantageously a peptide ligand-effector group or composition according to the invention will have a tβ half-life in the range 12 to 60 hours. In a further embodiment, it will have a tβ half-life of a day or more. In a further embodiment still, it will be in the range 12 to 26 hours.

In one particular embodiment of the invention, the functional group is selected from a metal chelator, which is suitable for complexing metal radioisotopes of medicinal relevance.

Possible effector groups also include enzymes, for instance such as carboxypeptidase G2 for use in enzyme/prodrug therapy, where the peptide ligand replaces antibodies in ADEPT.

In one particular embodiment of the invention, the functional group is selected from a drug, such as a cytotoxic agent for cancer therapy. Suitable examples include: alkylating agents such as cisplatin and carboplatin, as well as oxaliplatin, mechlorethamine, cyclophosphamide, chlorambucil, ifosfamide; Anti-metabolites including purine analogs azathioprine and mercaptopurine or pyrimidine analogs; plant alkaloids and terpenoids including vinca alkaloids such as Vincristine, Vinblastine, Vinorelbine and Vindesine; Podophyllotoxin and its derivatives etoposide and teniposide; Taxanes, including paclitaxel, originally known as Taxol; topoisomerase inhibitors including camptothecins: irinotecan and topotecan, and type II inhibitors including amsacrine, etoposide, etoposide phosphate, and teniposide. Further agents can include antitumour antibiotics which include the immunosuppressant dactinomycin (which is used in kidney transplantations), doxorubicin, epirubicin, bleomycin, calicheamycins, and others.

In one further particular embodiment of the invention, the cytotoxic agent is selected from maytansinoids (such as DM1) or monomethyl auristatins (such as MMAE).

DM1 is a cytotoxic agent which is a thiol-containing derivative of maytansine and has the following structure:

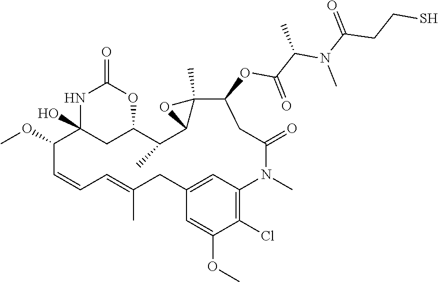 [SMILES: COc1cc2cc(c1Cl)N(C)C(=O)C[C@H](OC(=O)[C@H](C)N(C)C(=O)CCS)[C@]1(C)O[C@H]1[C@H](C)[C@@H]1C[C@@](O)(NC(=O)O1)[C@@H](OC)/C=C\C=C(/C)C2]

Monomethyl auristatin E (MMAE) is a synthetic antineoplastic agent and has the following structure:

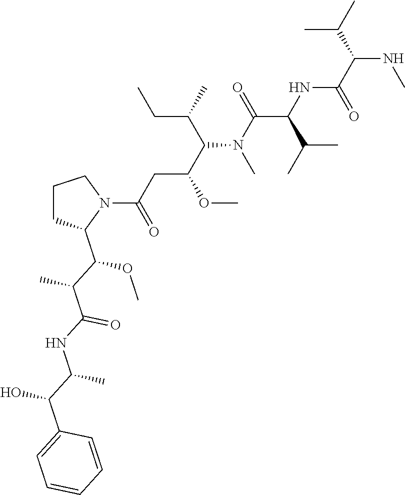

In one yet further particular embodiment of the invention, the cytotoxic agent is selected from monomethyl auristatin E (MMAE). Data is presented herein in FIG. 1 and Tables 4 and 5 which demonstrates the effects of peptide ligands conjugated to a toxin containing MMAE.

In one embodiment, the cytotoxic agent is linked to the bicyclic peptide by a cleavable bond, such as a disulphide bond or a protease sensitive bond. In a further embodiment, the groups adjacent to the disulphide bond are modified to control the hindrance of the disulphide bond, and by this the rate of cleavage and concomitant release of cytotoxic agent.

Published work established the potential for modifying the susceptibility of the disulphide bond to reduction by introducing steric hindrance on either side of the disulphide bond (Kellogg et al (2011) Bioconjugate Chemistry, 22, 717). A greater degree of steric hindrance reduces the rate of reduction by intracellular glutathione and also extracellular (systemic) reducing agents, consequentially reducing the ease by which toxin is released, both inside and outside the cell. Thus, selection of the optimum in disulphide stability in the circulation (which minimises undesirable side effects of the toxin) versus efficient release in the intracellular milieu (which maximises the therapeutic effect) can be achieved by careful selection of the degree of hindrance on either side of the disulphide bond.

The hindrance on either side of the disulphide bond is modulated through introducing one or more methyl groups on either the targeting entity (here, the bicyclic peptide) or toxin side of the molecular construct.

In one embodiment, the cytotoxic agent and linker is selected from any combinations of those described in WO 2016/067035 (the cytotoxic agents and linkers thereof are herein incorporated by reference).

In one embodiment, the linker between said cytotoxic agent and said bicyclic peptide comprises one or more amino acid residues. Examples of suitable amino acid residues as suitable linkers include Ala, Cit, Lys, Trp and Val.

In one embodiment, the cytotoxic agent is selected from MMAE and said drug conjugate additionally comprises a linker selected from: -PABC-Cit-Val-Glutaryl- or -PABC-cyclobutyl-Ala-Cit-βAla-, wherein PABC represents p-aminobenzylcarbamate. Full details of the cyclobutyl containing linker may be found in Wei et al (2018) J. Med. Chem. 61, 989-1000. In a further embodiment, the cytotoxic agent is selected from MMAE and the linker is -PABC-Cit-Val-Glutaryl-.

In one embodiment, the cytotoxic agent is MMAE, the bicyclic peptide is selected from (B-Ala)-Sar10-ALPP-(SEQ ID NO: 17) and the linker is selected from -PABC-Cit-Val-Glutaryl-. This BDC is known herein as BT17BDC58 which is represented schematically as:

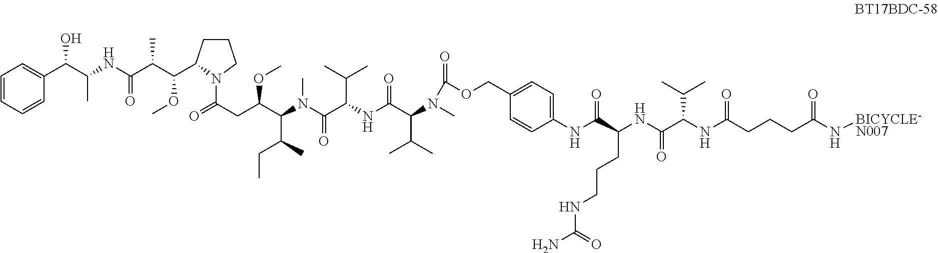

(wherein BICYCLE-N007 represents (B-Ala)-Sar10-ALPP-(SEQ ID NO: 17) also known as (B-Ala)-Sar10-A-(17-120-09-T03) HArg2 HArg9).

Data is presented herein in FIG. 1 and Tables 4 and 5 which demonstrates dose-dependent antitumor activity.

Synthesis

The peptides of the present invention may be manufactured synthetically by standard techniques followed by reaction with a molecular scaffold in vitro. When this is performed, standard chemistry may be used. This enables the rapid large scale preparation of soluble material for further downstream experiments or validation. Such methods could be accomplished using conventional chemistry such as that disclosed in Timmerman et al (supra).

Thus, the invention also relates to manufacture of polypeptides selected as set out herein, wherein the manufacture comprises optional further steps as explained below. In one embodiment, these steps are carried out on the end product polypeptide made by chemical synthesis.

Peptides can also be extended, to incorporate for example another loop and therefore introduce multiple specificities.

To extend the peptide, it may simply be extended chemically at its N-terminus or C-terminus or within the loops using orthogonally protected lysines (and analogues) using standard solid phase or solution phase chemistry. Standard (bio)conjugation techniques may be used to introduce an activated or activatable N- or C-terminus. Alternatively additions may be made by fragment condensation or native chemical ligation e.g. as described in (Dawson et al. 1994. Synthesis of Proteins by Native Chemical Ligation. Science 266:776-779), or by enzymes, for example using subtiligase as described in (Chang et al. Proc Natl Acad Sci USA. 1994 Dec. 20; 91(26):12544-8 or in Hikari et al Bioorganic & Medicinal Chemistry Letters Volume 18, Issue 22, 15 Nov. 2008, Pages 6000-6003).

Alternatively, the peptides may be extended or modified by further conjugation through disulphide bonds. This has the additional advantage of allowing the first and second peptide to dissociate from each other once within the reducing environment of the cell. In this case, the molecular scaffold (e.g. TATA) could be added during the chemical synthesis of the first peptide so as to react with the three cysteine groups; a further cysteine or thiol could then be appended to the N or C-terminus of the first peptide, so that this cysteine or thiol only reacted with a free cysteine or thiol of the second peptide, forming a disulfide-linked bicyclic peptide-peptide conjugate.

Similar techniques apply equally to the synthesis/coupling of two bicyclic and bispecific macrocycles, potentially creating a tetraspecific molecule.

Furthermore, addition of other functional groups or effector groups may be accomplished in the same manner, using appropriate chemistry, coupling at the N- or C-termini or via side chains. In one embodiment, the coupling is conducted in such a manner that it does not block the activity of either entity.

Pharmaceutical Compositions

According to a further aspect of the invention, there is provided a pharmaceutical composition comprising a peptide ligand as defined herein in combination with one or more pharmaceutically acceptable excipients.

Generally, the present peptide ligands will be utilised in purified form together with pharmacologically appropriate excipients or carriers. Typically, these excipients or carriers include aqueous or alcoholic/aqueous solutions, emulsions or suspensions, including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The compounds of the invention can be used alone or in combination with another agent or agents. The other agent for use in combination may be for example another antibiotic, or an antibiotic ‘adjuvant’ such as an agent for improving permeability into Gram-negative bacteria, an inhibitor of resistance determinants or an inhibitor of virulence mechanisms.

Suitable antibiotics for use in combination with the compounds of the invention include but are not limited to:

Beta lactams, such as penicillins, cephalosporins, carbapenems or monobactams. Suitable penicillins include oxacillin, methicillin, ampicillin, cloxacillin, carbenicillin, piperacillin, tricarcillin, flucloxacillin, and nafcillin; suitable cephalosporins include cefazolin, cefalexin, cefalothin, ceftazidime, cefepime, ceftobiprole, ceftaroline, ceftolozane and cefiderocol; suitable carbapenems include meropenem, doripenem, imipenem, ertapenem, biapenem and tebipenem; suitable monobactams include aztreonam;

Lincosamides such as clindamycin and lincomycin;

Macrolides such as azithromycin, clarithromycin, erythromycin, telithromycin and solithromycin;

Tetracyclines such as tigecycline, omadacycline, eravacycline, doxycycline, and minocycline;

Quinolones such as ciprofloxacin, levofloxacin, moxifloxacin, and delafloxacin;

Rifamycins such as rifampicin, rifabutin, rifalazil, rifapentine, and rifaximin;

Aminoglycosides such as gentamycin, streptomycin, tobramycin, amikacin and plazomicin;

Glycopeptides such as vancomycin, teichoplanin, telavancin, dalbavancin, and oritavancin, Pleuromutilins such as lefamulin

Oxazolidinones such as linezolid or tedizolid

Polymyxins such as polymyxin B or colistin;

Trimethoprim, iclaprim, sulfamethoxazole;

Metronidazole;

Fidaxomicin:

Mupirocin;

Fusidic acid;

Daptomycin;

Murepavidin;

Fosfomycin; and

Nitrofurantoin.

Suitable antibiotic ‘adjuvants’ include but are not limited to:

agents known to improve uptake into bacteria such as outer membrane permeabilisers or efflux pump inhibitors; outer membrane permeabilisers may include polymyxin B nonapeptide or other polymyxin analogues, or sodium edetate;

inhibitors of resistance mechanisms such as beta-lactamase inhibitors; suitable beta-lactamase inhibitors include clavulanic acid, tazobactam, sulbactam, avibactam, relebactam and nacubactam; and

inhibitors of virulence mechanisms such as toxins and secretion systems, including antibodies.

The compounds of the invention can also be used in combination with biological therapies such as nucleic acid based therapies, antibodies, bacteriophage or phage lysins.

The route of administration of pharmaceutical compositions according to the invention may be any of those commonly known to those of ordinary skill in the art. For therapy, the peptide ligands of the invention can be administered to any patient in accordance with standard techniques. Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly. Preferably, the pharmaceutical compositions according to the invention will be administered parenterally. The dosage and frequency of administration will depend on the age, sex and condition of the patient, concurrent administration of other drugs, counterindications and other parameters to be taken into account by the clinician.

The peptide ligands of this invention can be lyophilised for storage and reconstituted in a suitable carrier prior to use. This technique has been shown to be effective and art-known lyophilisation and reconstitution techniques can be employed. It will be appreciated by those skilled in the art that lyophilisation and reconstitution can lead to varying degrees of activity loss and that levels may have to be adjusted upward to compensate.

The compositions containing the present peptide ligands or a cocktail thereof can be administered for therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 10 μg to 250 mg of selected peptide ligand per kilogram of body weight, with doses of between 100 μg to 25 mg/kg/dose being more commonly used.

A composition containing a peptide ligand according to the present invention may be utilised in therapeutic settings to treat a microbial infection or to provide prophylaxis to a subject at risk of infection eg undergoing surgery, chemotherapy, artificial ventilation or other condition or planned intervention. In addition, the peptide ligands described herein may be used extracorporeally or in vitro selectively to kill, deplete or otherwise effectively remove a target cell population from a heterogeneous collection of cells. Blood from a mammal may be combined extracorporeally with the selected peptide ligands whereby the undesired cells are killed or otherwise removed from the blood for return to the mammal in accordance with standard techniques.

Therapeutic Uses

The bicyclic peptides of the invention have specific utility as high affinity binders of membrane type 1 metalloprotease (MT1-MMP, also known as MMP14). MT1-MMP is a transmembrane metalloprotease that plays a major role in the extracellular matrix remodeling, directly by degrading several of its components and indirectly by activating pro-MMP2. MT1-MMP is crucial for tumor angiogenesis (Sounni et al (2002) FASEB J. 16(6), 555-564) and is over-expressed on a variety of solid tumours, therefore the drug conjugates comprising MT1-MMP-binding bicycle peptides of the present invention have particular utility in the targeted treatment of cancer, in particular solid tumours such as non-small cell lung carcinomas. In one embodiment, the bicyclic peptide of the invention is specific for human MT1-MMP. In a further embodiment, the bicyclic peptide of the invention is specific for mouse MT1-MMP. In a yet further embodiment, the bicyclic peptide of the invention is specific for human and mouse MT1-MMP. In a yet further embodiment, the bicyclic peptide of the invention is specific for human, mouse and dog MT1-MMP.

Polypeptide ligands of the invention may be employed in in vivo therapeutic and prophylactic applications, in vitro and in vivo diagnostic applications, in vitro assay and reagent applications, and the like. Ligands having selected levels of specificity are useful in applications which involve testing in non-human animals, where cross-reactivity is desirable, or in diagnostic applications, where cross-reactivity with homologues or paralogues needs to be carefully controlled. In some applications, such as vaccine applications, the ability to elicit an immune response to predetermined ranges of antigens can be exploited to tailor a vaccine to specific diseases and pathogens.

Substantially pure peptide ligands of at least 90 to 95% homogeneity are preferred for administration to a mammal, and 98 to 99% or more homogeneity is most preferred for pharmaceutical uses, especially when the mammal is a human. Once purified, partially or to homogeneity as desired, the selected polypeptides may be used diagnostically or therapeutically (including extracorporeally) or in developing and performing assay procedures, immunofluorescent stainings and the like (Lefkovite and Pernis, (1979 and 1981) Immunological Methods, Volumes I and II, Academic Press, NY).

The conjugates of the peptide ligands of the present invention will typically find use in preventing, suppressing or treating cancer, in particular solid tumours such as non-small cell lung carcinomas.

Thus, according to a further aspect of the invention, there are provided drug conjugates of the peptide ligand as defined herein for use in preventing, suppressing or treating cancer, in particular solid tumours such as non-small cell lung carcinomas.

According to a further aspect of the invention, there is provided a method of preventing, suppressing or treating cancer, in particular solid tumours such as non-small cell lung carcinomas which comprises administering to a patient in need thereof a drug conjugate of the peptide ligand as defined herein.

Examples of cancers (and their benign counterparts) which may be treated (or inhibited) include, but are not limited to tumours of epithelial origin (adenomas and carcinomas of various types including adenocarcinomas, squamous carcinomas, transitional cell carcinomas and other carcinomas) such as carcinomas of the bladder and urinary tract, breast, gastrointestinal tract (including the esophagus, stomach (gastric), small intestine, colon, rectum and anus), liver (hepatocellular carcinoma), gall bladder and biliary system, exocrine pancreas, kidney, lung (for example adenocarcinomas, small cell lung carcinomas, non-small cell lung carcinomas, bronchioalveolar carcinomas and mesotheliomas), head and neck (for example cancers of the tongue, buccal cavity, larynx, pharynx, nasopharynx, tonsil, salivary glands, nasal cavity and paranasal sinuses), ovary, fallopian tubes, peritoneum, vagina, vulva, penis, cervix, myometrium, endometrium, thyroid (for example thyroid follicular carcinoma), adrenal, prostate, skin and adnexae (for example melanoma, basal cell carcinoma, squamous cell carcinoma, keratoacanthoma, dysplastic naevus); haematological malignancies (i.e. leukemias, lymphomas) and premalignant haematological disorders and disorders of borderline malignancy including haematological malignancies and related conditions of lymphoid lineage (for example acute lymphocytic leukemia [ALL], chronic lymphocytic leukemia [CLL], B-cell lymphomas such as diffuse large B-cell lymphoma [DLBCL], follicular lymphoma, Burkitt's lymphoma, mantle cell lymphoma, T-cell lymphomas and leukaemias, natural killer [NK] cell lymphomas, Hodgkin's lymphomas, hairy cell leukaemia, monoclonal gammopathy of uncertain significance, plasmacytoma, multiple myeloma, and post-transplant lymphoproliferative disorders), and haematological malignancies and related conditions of myeloid lineage (for example acute myelogenousleukemia [AML], chronic myelogenousleukemia [CML], chronic myelomonocyticleukemia [CMML], hypereosinophilic syndrome, myeloproliferative disorders such as polycythaemia vera, essential thrombocythaemia and primary myelofibrosis, myeloproliferative syndrome, myelodysplastic syndrome, and promyelocyticleukemia);

tumours of mesenchymal origin, for example sarcomas of soft tissue, bone or cartilage such as osteosarcomas, fibrosarcomas, chondrosarcomas, rhabdomyosarcomas, leiomyosarcomas, liposarcomas, angiosarcomas, Kaposi's sarcoma, Ewing's sarcoma, synovial sarcomas, epithelioid sarcomas, gastrointestinal stromal tumours, benign and malignant histiocytomas, and dermatofibrosarcomaprotuberans; tumours of the central or peripheral nervous system (for example astrocytomas, gliomas and glioblastomas, meningiomas, ependymomas, pineal tumours and schwannomas); endocrine tumours (for example pituitary tumours, adrenal tumours, islet cell tumours, parathyroid tumours, carcinoid tumours and medullary carcinoma of the thyroid); ocular and adnexal tumours (for example retinoblastoma); germ cell and trophoblastic tumours (for example teratomas, seminomas, dysgerminomas, hydatidiform moles and choriocarcinomas); and paediatric and embryonal tumours (for example medulloblastoma, neuroblastoma, Wilms tumour, and primitive neuroectodermal tumours); or syndromes, congenital or otherwise, which leave the patient susceptible to malignancy (for example Xeroderma Pigmentosum).

References herein to the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest.

Animal model systems which can be used to screen the effectiveness of the drug conjugates in protecting against or treating the disease are available. The use of animal model systems is facilitated by the present invention, which allows the development of polypeptide ligands which can cross react with human and animal targets, to allow the use of animal models.

The invention is further described below with reference to the following examples.

### Examples

Materials and Methods

Peptide Synthesis

Peptide synthesis was based on Fmoc chemistry, using a Symphony peptide synthesiser manufactured by Peptide Instruments and a Syro II synthesiser by MultiSynTech. Standard Fmoc-amino acids were employed (Sigma, Merck), with appropriate side chain protecting groups: where applicable standard coupling conditions were used in each case, followed by deprotection using standard methodology.

Alternatively, peptides were purified using HPLC and following isolation they were modified with 1,3,5-triacryloylhexahydro-1,3,5-triazine (TATA, Sigma). For this, linear peptide was diluted with 50:50 MeCN:H₂O up to ˜35 mL, ˜500 μL of 100 mM TATA in acetonitrile was added, and the reaction was initiated with 5 mL of 1 M NH₄HCO₃ in H₂O. The reaction was allowed to proceed for ˜30-60 min at RT, and lyophilised once the reaction had completed (judged by MALDI). Once completed, 1 ml of 1M L-cysteine hydrochloride monohydrate (Sigma) in H₂O was added to the reaction for ˜60 min at RT to quench any excess TATA. Following lyophilisation, the modified peptide was purified as above, while replacing the Luna C8 with a Gemini C18 column (Phenomenex), and changing the acid to 0.1% trifluoroacetic acid. Pure fractions containing the correct TATA-modified material were pooled, lyophilised and kept at −20° C. for storage.

All amino acids, unless noted otherwise, were used in the L-configurations.

In some cases peptides are converted to activated disulfides prior to coupling with the free thiol group of a toxin using the following method; a solution of 4-methyl(succinimidyl 4-(2-pyridylthio)pentanoate) (100 mM) in dry DMSO (1.25 mol equiv) was added to a solution of peptide (20 mM) in dry DMSO (1 mol equiv). The reaction was well mixed and DIPEA (20 mol equiv) was added. The reaction was monitored by LC/MS until complete.

Preparation of Bicyclic Drug Conjugate BT17BDC58

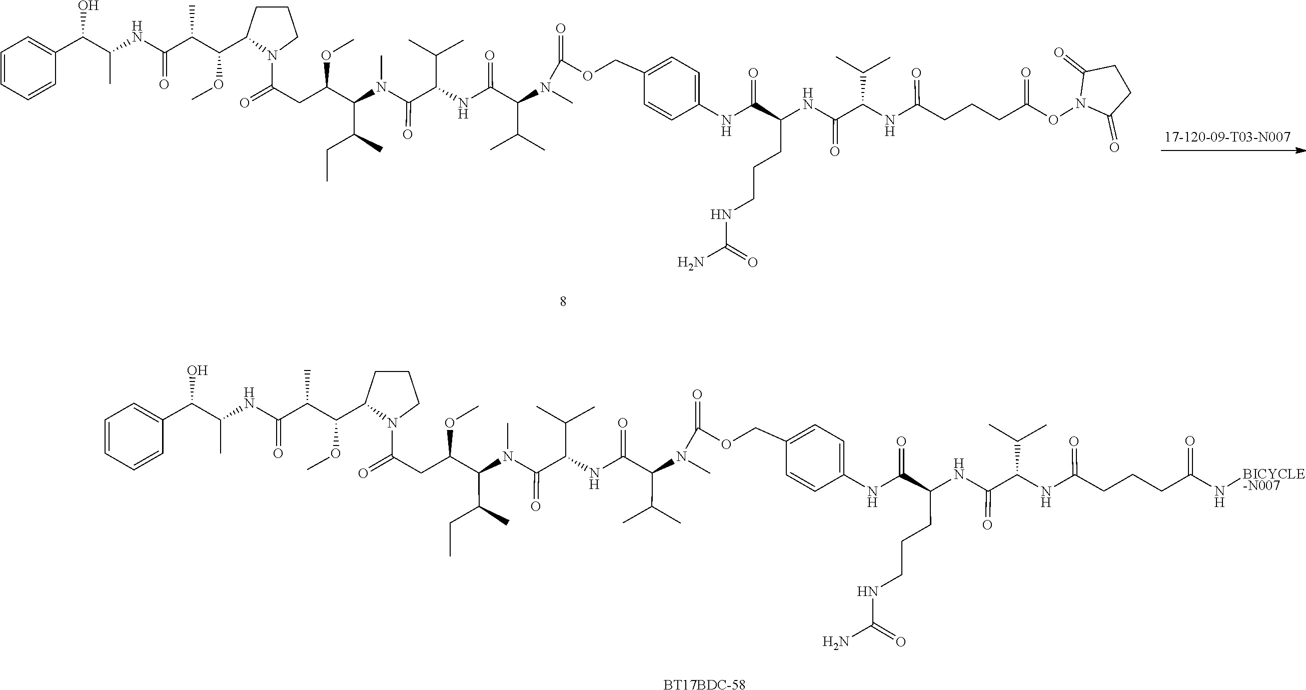

A 50 mL round bottom flask which contained BICYCLE-NH₂ ((B-Ala)-Sar10-ALPP-(SEQ ID NO: 17), also known as (B-Ala)-Sar10-A-(17-120-09-T03) HArg2 HArg9) (66 mg, 22.4 μmol, 1.00 eq) in DMA (5 mL) was purged using nitrogen balloon. DIEA (2.91 mg, 112.4 μmol, 19.6 uL, 5 eq) was then added with stirring at 25° C. Compound 8 (which may be prepared according to the method described for Compound 8 in WO 2018/127699) (30.00 mg, 22.48 μmol, 1.00 eq) was then added and the reaction was stirred under a positive nitrogen atmosphere at 25° C. for 16 hrs. LC-MS showed Compound 8 was consumed completely and one main peak with desired MS was detected. The resulting reaction mixture was purified by prep-HPLC (TFA condition). Compound BT17BDC58 (20.2 mg, 4.85 μmol, 21.56% yield) was obtained as a white solid.

Biological Data

MT1-MMP Fluorescence Polarisation Competition Binding Assay

Due to their high affinities to the MT1-MMP Hemopexin domain (PEX), the fluoresceinated derivatives of 17-69-07 and 17-69-12 (denoted as 17-69-07-N040, 17-69-07-N041 and 17-69-12-N004) can be used for competition experiments (using FP for detection)

Here, a pre-formed complex of PEX with the fluorescent PEX-binding tracer is titrated with free, non-fluoresceinated bicyclic peptide. Since all 17-69-based peptides are expected to bind at the same site, the titrant will displace the fluorescent tracer from PEX. Dissociation of the complex can be measured quantitatively, and the Kd of the competitor (titrant) to the target protein determined. The advantage of the competition method is that the affinities of non-fluoresceinated bicyclic peptides can be determined accurately and rapidly.

Concentrations of tracer are usually at the Kd or below (here, 1 nM), and the binding protein (here, hemopexin of MT1-MMP) is at a 15-fold excess such that >90% of the tracer is bound. Subsequently, the non-fluorescent competitor bicyclic peptide (usually just the bicycle core sequence) is titrated, such that it displaces the fluorescent tracer from the target protein. The displacement of the tracer is measured and associated with a drop in fluorescence polarisation. The drop in fluorescence polarisation is proportional to the fraction of target protein bound with the non-fluorescent titrant, and thus is a measure of the affinity of titrant to target protein.

In certain experiments, collagen binding tracers (i.e. 17-88-N006 and 17-88-226-N002) were used in an analogous manner to the hemopexin binding tracers.

The raw data is fit to the analytical solution of the cubic equation that describes the equilibria between fluorescent tracer, titrant, and binding protein. The fit requires the value of the affinity of fluorescent tracer to the target protein, which can be determined separately by direct binding FP experiments (see previous section). The curve fitting was performed using Sigmaplot 12.0 and used an adapted version of the equation described by Zhi-Xin Wang (FEBS Letters 360 (1995) 111-114).

TABLE 1
Characterising Data for Tracers Used
in Fluorescence Polarisation
Competition Assay

| | | | Kd (nM) | |
|---|---|---|---|---|
| | | | (Direct | Binding |
| Name | Sequence | Scaffold | Binding) | Site |
| 17-69-07- | ACYN | TBMB | 0.52 | Hemopexin |
| N040 | EFGC | | | |
| | EDFY | | | |
| | DICA | | | |
| | [Sar]₆ | | | |
| | [KFI] | | | |
| | ((SEQ ID | | | |
| | NO: 140)- | | | |
| | [Sar]₆ | | | |
| | [KFI]) | | | |
| 17-69-12- | [FI]G | TBMB | 1.0 | Hemopexin |
| N004 | [Sar]₅ | | | |
| | ACMNQ | | | |
| | FGCED | | | |
| | FYDICA | | | |
| | ([FI]G | | | |
| | [Sar]₅- | | | |
| | (SEQ ID | | | |
| | NO: 141)) | | | |
| 17-69-07- | [FI]G | TBMB | 3.4 | Hemopexin |
| N041 | [Sar]₅ | | | |
| | ACYNEF | | | |
| | ACED | | | |
| | FYDICA | | | |
| | ([FI]G | | | |
| | [Sar]5- | | | |
| | (SEQ ID | | | |
| | NO: 142)) | | | |
| 17-88- | ACPYSWET | TBMB | 14 | Collagen |
| N006 | CLFGDYRC | | | |
| | A[Sar]₆ | | | |
| | [KFI] | | | |
| | ((SEQ ID | | | |
| | NO: 143)- | | | |
| | [Sar]₆ | | | |
| | [KFI]) | | | |
| 17-88- | ACPYD | TBMB | 50 | Collagen |
| 226-N002 | WATC | | | |
| | LFGD | | | |
| | YRCA | | | |
| | [Sar]₆ | | | |
| | [KFI] | | | |
| | ((SEQ ID | | | |
| | NO: 144)- | | | |
| | [Sar]₆ | | | |
| | [KFI]) | | | |

Certain peptide ligands of the invention were tested in the above mentioned binding assay and the results are shown in Table 2:

TABLE 2
Competition Binding Data for Selected Peptide Ligands of the Invention

| Bicycle Name | Tracer | Kd (nM ± 95% CI) | |
|---|---|---|---|
| 17-108-02 | 17-88-N006 | 4488.4 ± 545.85 | |
| 17-111-01 | 17-69-07-N041 | 1800 | n = 1 |
| 17-111-02 | 17-69-07-N041 | 2300 | n = 1 |
| 17-116-01 | 17-69-07-N040 | 352 | n = 1 |
| 17-120-00 | 17-88-N006 | 923 ± 287.43 | |
| 17-120-01 | 17-88-N006 | 310.33 ± 30.89 | |
| 17-120-02 | 17-88-N006 | 190.2 ± 37.57 | |
| 17-120-03 | 17-88-N006 | 603.56 ± 35 | |
| 17-120-04 | 17-88-N006 | 224.5 | n = 1 |
| 17-120-05 | 17-69-07-N040 | >279.5 ± 69.3 | |
| 17-120-07 | 17-88-N006 | 273 ± 119.56 | |
| 17-120-08 | 17-88-N006 | 258 ± 101.92 | |
| 17-120-09-T01 | 17-88-N006 | 53.83 ± 13.33 | |
| 17-120-09-T02 | 17-88-N006 | 55.2 | n = 1 |
| 17-120-09-T03 (BCY1124) | 17-88-N006 | 35.6 | n = 1 |
| 17-120-09-T03 (BCY1124) | 17-88-226-N002 | 32 ± 20.24 | |
| Ac-(17-120-09-T03) (BCY1125) | 17-88-226-N002 | 18.65 ± 6.96 | |
| Sar3-A-(17-120-09-T03) | 17-88-226-N002 | 16.33 ± 5.73 | |
| Sar3-A-(17-120-09-T03) HArg2 | 17-88-226-N002 | 26.63 ± 9.31 | |
| Sar3-A-(17-120-09-T03) Arg9 | 17-88-226-N002 | 8.28 ± 3.53 | |
| Sar3-A-(17-120-09-T03) HArg9 | 17-88-226-N002 | 30.9 ± 10 | |
| (B-Ala)-Sar10-A-(17-120-09-T03) HArg2 HArg9 | 17-88-226-N002 | 39.5 ± 16.43 | |
| (B-Ala)-Sar10-A-(17-120-09-T03) HArg2 Ala9 | 17-88-226-N002 | 189.55 ± 32.24 | |
| (B-Ala)-Sar10-A-(17-120-09-T03) Ala2 HArg9 | 17-88-226-N002 | 89.55 ± 10.09 | |
| Ac-(B-Ala)-Sar10-A-(17-120-09-T03) HArg2 HArg9 | 17-88-226-N002 | 43.9 ± 15.48 | |
| 17-120-09-T04 | 17-88-N006 | 34.15 ± 10.2 | |
| 17-120-09-T05 | 17-88-N006 | 32.3 ± 6.81 | |
| 17-120-09-T06 | 17-88-N006 | 49.8 | n = 1 |
| 17-120-09-T07 | 17-88-N006 | 48.1 | n = 1 |
| 17-120-09-T08 | 17-88-N006 | 37.5 | n = 1 |
| 17-120-09-T09 | 17-88-N006 | 77.2 | n = 1 |
| 17-120-09-T10 | 17-88-N006 | 38.5 | n = 1 |
| 17-120-09-T11 | 17-88-N006 | 44.2 | n = 1 |
| 17-120-09-T12 | 17-88-N006 | 62.2 | n = 1 |
| 17-120-09-T13 | 17-88-N006 | 69.3 | n = 1 |
| 17-120-10-T01 | 17-88-N006 | 132.3 ± 103.29 | |
| 17-120-11-T01 | 17-88-N006 | 612 ± 760.47 | |
| 17-120-12-T01 | 17-88-N006 | 183 | n = 1 |
| 17-120-13-T01 | 17-88-N006 | 189 ± 123.48 | |
| 17-120-14-T01 | 17-88-N006 | 148 | n = 1 |
| 17-120-15-T01 | 17-88-N006 | 178 | n = 1 |
| 17-120-15-T02 | 17-88-N006 | 76.67 ± 72.87 | |
| 17-120-16-T01 | 17-88-N006 | 74.4 ± 17.64 | |
| 17-120-17-T01 | 17-88-N006 | 157 | n = 1 |
| 17-120-18 | 17-88-N006 | 252 ± 92.12 | |
| 17-120-19 | 17-88-N006 | 303 ± 258.72 | |
| 17-120-20 | 17-88-N006 | 248.5 ± 14.7 | |
| 17-120-21 | 17-88-N006 | >82.75 ± 4.61 | |
| 17-120-21-T01 | 17-88-N006 | 113 | n = 1 |
| 17-120-22-T01 | 17-88-N006 | 62.35 ± 30.48 | |
| 17-120-22-T02 | 17-88-N006 | 46.1 ± 26.5 | |
| 17-120-23-T01 | 17-88-N006 | 127 ± 7.84 | |
| 17-120-24-T01 | 17-88-N006 | 126 ± 35.28 | |
| 17-120-25-T01 | 17-88-N006 | 194.5 ± 161.7 | |
| 17-120-26-T01 | 17-88-N006 | 598 | n = 1 |
| 17-120-27-T01 | 17-88-N006 | 394 | n = 1 |
| 17-120-28-T01 | 17-88-N006 | 191.5 ± 4.9 | |
| 17-120-29-T01 | 17-88-N006 | 162 ± 68.6 | |
| 17-120-30-T01 | 17-88-N006 | 78.7 | n = 1 |
| 17-120-31-T01 | 17-88-N006 | 50.2 | n = 1 |
| 17-120-31-T02 | 17-88-N006 | 68.3 | n = 1 |
| 17-120-31-T03 | 17-88-N006 | 41.47 ± 5.32 | |
| 17-120-32-T01 | 17-88-N006 | 63.8 ± 26.49 | |
| 17-120-33-T01 | 17-88-N006 | 77.6 | n = 1 |
| 17-120-34-T01 | 17-88-N006 | 59.87 ± 8.58 | |
| 17-120-35-T01 | 17-88-N006 | 23.33 ± 9.48 | |
| 17-121-00 | 17-69-07-N040 | 678 | n = 1 |
| 17-122-02 | 17-69-07-N040 | >929 | n = 3 |
| 17-122-03 | 17-69-07-N040 | >378 | n = 3 |
| 17-122-04 | 17-69-07-N040 | >2100 | |
| 17-126-01 | 17-69-07-N041 | >316 ± 9.8 | |
| 17-126-02 | 17-69-07-N041 | >282 ± 172.48 | |
| 17-126-03 | 17-69-07-N041 | >430 ± 11.76 | |
| 17-126-06 | 17-69-07-N040 | 675 ± 192.08 | |
| 17-126-07 | 17-69-07-N040 | 197.5 ± 85.26 | |
| 17-126-08 | 17-69-07-N040 | 711 | n = 1 |
| 17-126-09 | 17-69-07-N040 | 165 | n = 1 |
| 17-126-10 | 17-69-07-N040 | 737 | n = 1 |
| 17-126-11 | 17-69-07-N040 | 971 | n = 1 |
| 17-126-12 | 17-69-07-N040 | 2900 | n = 1 |
| 17-126-18 | 17-69-07-N040 | 147 | n = 1 |
| 17-126-19 | 17-69-07-N040 | 199 | n = 1 |
| 17-126-20 | 17-69-07-N040 | 246 | n = 1 |
| 17-126-21 | 17-69-07-N040 | 131 | n = 1 |
| 17-126-22 | 17-69-07-N040 | 295 | n = 1 |
| 17-126-23 | 17-69-07-N040 | 409 | n = 1 |
| 17-126-24 | 17-69-07-N040 | 200 | n = 1 |
| 17-126-25 | 17-69-07-N040 | >138.76 | |
| Ac-(17-126-25) | 17-69-12-N004 | 60.5 | n = 1 |
| 17-126-26 | 17-69-07-N040 | 1200 | n = 1 |
| 17-126-27 | 17-69-07-N040 | 143 | n = 1 |
| 17-126-28 | 17-69-07-N040 | 250 | n = 1 |
| 17-126-30-T01 | 17-69-07-N040 | 239 | n = 1 |
| 17-126-31-T01 | 17-69-07-N040 | 295 | n = 1 |
| 17-126-32-T01 | 17-69-07-N040 | 390 | n = 1 |
| 17-126-33-T01 | 17-69-07-N040 | 244 | n = 1 |
| 17-126-33-T02 | 17-69-07-N040 | 296 | n = 1 |
| 17-126-35-T01 | 17-69-07-N040 | 263 | n = 1 |
| 17-126-36-T01 | 17-69-07-N040 | 149 | n = 1 |
| 17-126-37-T01 | 17-69-07-N040 | 155 | n = 1 |
| 17-126-38-T01 | 17-69-07-N040 | 162 | n = 1 |
| 17-126-39-T01 | 17-69-07-N040 | 187 | n = 1 |
| 17-126-40-T01 | 17-69-07-N040 | 310 | n = 1 |
| 17-126-41-T01 | 17-69-07-N040 | 202 | n = 1 |
| 17-127-01 | 17-69-07-N040 | 2200 | n = 1 |
| 17-129-00 | 17-69-07-N040 | 446.0 | |
| 17-129-01-T01 | 17-69-07-N040 | 499 | n = 1 |
| 17-129-01-T02 | 17-69-07-N040 | 525 | n = 1 |
| 17-129-01-T03 | 17-69-07-N040 | 598 | n = 1 |
| 17-129-01-T04 | 17-69-07-N040 | 705 | n = 1 |
| 17-129-01-T05 | 17-69-07-N040 | 324 | n = 1 |
| 17-129-02 | 17-69-07-N040 | 877 ± 650.71 | |
| 17-129-03 | 17-69-07-N040 | 536.5 ± 126.42 | |
| 17-129-04 | 17-69-07-N040 | 595 ± 372.39 | |
| 17-129-05 | 17-69-07-N040 | 136.17 ± 22.27 | |
| 17-129-06 | 17-69-07-N040 | 566 | n = 1 |
| 17-129-07 | 17-69-07-N040 | 582 | n = 1 |
| 17-129-08 | 17-69-07-N040 | 516 | n = 1 |
| 17-129-09 | 17-69-07-N040 | 1092 | n = 1 |
| 17-129-10 | 17-69-07-N040 | 781 | n = 1 |
| 17-129-11 | 17-69-07-N040 | 912 | n = 1 |
| 17-129-12-T01 | 17-69-07-N040 | 187 ± 86.24 | |
| 17-129-13-T01 | 17-69-07-N040 | 248 | n = 1 |
| 17-129-14-T01 | 17-69-07-N040 | 245.5 ± 46.06 | |
| 17-129-14-T02 | 17-69-07-N040 | 318 ± 27.44 | |
| 17-129-15-T01 | 17-69-07-N040 | 278 | n = 1 |
| 17-129-16-T01 | 17-69-07-N040 | 263 | n = 1 |
| 17-129-17-T01 | 17-69-07-N040 | 418 | n = 1 |
| 17-129-17-T02 | 17-69-07-N040 | 369 | n = 1 |
| 17-129-17-T03 | 17-69-07-N040 | 312 | n = 1 |
| 17-129-18-T01 | 17-69-07-N040 | 138.33 ± 43.96 | |
| 17-129-18-T02 | 17-69-07-N040 | 334 | n = 1 |
| 17-129-18-T03 | 17-69-07-N040 | 202 ± 92.96 | |
| 17-129-18-T04 | 17-69-07-N040 | 171.5 ± 53.9 | |
| 17-129-19-T01 | 17-69-07-N040 | 754 | n = 1 |
| 17-129-19-T02 | 17-69-07-N040 | 458 | n = 1 |
| 17-129-20-T01 | 17-69-07-N040 | 213 | n = 1 |
| 17-129-21-T01 | 17-69-07-N040 | 110.8 ± 33.71 | |
| 17-129-22-T01 | 17-69-07-N040 | 53.7 | |
| 17-129-23-T01 | 17-69-07-N040 | 54.8 | |
| Ac-(17-129-23-T01) | 17-69-12-N004 | 8.9 | n = 1 |

SPR Binding Data

Biacore experiments were performed to determine K_{D} (nM) values of monomeric peptides binding to human MT1 MMP14 protein, hemopexin domain (obtained from Merck Millipore).

The protein was randomly biotinylated in PBS using EZ-Link™ Sulfo-NHS-LC-LC-Biotin reagent (Thermo Fisher) as per the manufacturer's suggested protocol. The protein was extensively desalted to remove uncoupled biotin using spin columns into PBS.

For analysis of peptide binding, a Biacore 3000 instrument was used utilising a CM5 chip (GE Healthcare). Streptavidin was immobilized on the chip using standard amine-coupling chemistry at 25° C. with HBS-N (10 mM HEPES, 0.15 M NaCl, pH 7.4) as the running buffer. Briefly, the carboxymethyl dextran surface was activated with a 7 minute injection of a 1:1 ratio of 0.4 M 1-ethyl-3-(3-dimethylaminopropyl) carbodiimide hydrochloride (EDC)/0.1 M N-hydroxy succinimide (NHS) at a flow rate of 10 μl/min. For capture of streptavidin, the protein was diluted to 0.2 mg/ml in 10 mM sodium acetate (pH 4.5) and captured by injecting 120 μl of streptavidin onto the activated chip surface. Residual activated groups were blocked with a 7 minute injection of 1 M ethanolamine (pH 8.5) and biotinylated MT1 MMP14 captured to a level of 1,200-1,800 RU. Buffer was changed to PBS/0.05% Tween 20 and a dilution series of the peptides was prepared in this buffer with a final DMSO concentration of 0.5%. The top peptide concentration was 100 nM with 6 further 2-fold dilutions. The SPR analysis was run at 25° C. at a flow rate of 50 μl/min with 60 seconds association and dissociation between 400 and 1,200 seconds depending upon the individual peptide. Data were corrected for DMSO excluded volume effects. All data were double-referenced for blank injections and reference surface using standard processing procedures and data processing and kinetic fitting were performed using Scrubber software, version 2.0c (BioLogic Software). Data were fitted using a simple 1:1 binding model allowing for mass transport effects where appropriate.

Certain peptide ligands of the invention were tested in the above mentioned SPR and competition binding assays and the results are shown in Table 3:

TABLE 3
SPR and Competition Binding Data for Selected
Peptide Ligands of the Invention

| | | Plate 1 | Plate 2 |
|---|---|---|---|
| | KD (SPR)/ | Ki (FP-comp)/ | Ki (FP-comp)/ |
| Bicycle Name | nM | nM | nM |
| BCY1124 | 14.8 | 78 | 107 |
| BCY1125 | 15.1 | ~ | ~ |
| BCY3959 | 27.7 | ~ | ~ |
| BCY9933 | 30.7 | ~ | ~ |
| BCY9934 | 36.9 | ~ | ~ |
| BCY9935 | 39.2 | ~ | ~ |
| BCY9936 | 38 | ~ | ~ |
| BCY9937 | 77.8 | ~ | ~ |
| BCY9938 | 80.9 | ~ | ~ |
| BCY9943 | 299 | ~ | ~ |
| BCY9945 | 1360 | ~ | ~ |
| BCY9946 | 372 | ~ | ~ |
| BCY9949 | 190 | ~ | ~ |
| BCY9951 | 364 | ~ | ~ |
| BCY9952 | 870 | ~ | ~ |
| BCY9953 | 296 | ~ | ~ |
| BCY9954 | 28.3 | ~ | ~ |
| BCY9955 | 73.5 | ~ | ~ |
| BCY9957 | 304.2 | ~ | ~ |
| BCY9959 | 5.87 | ~ | ~ |
| BCY9960 | 72.7 | ~ | ~ |
| BCY9961 | 13000 | ~ | ~ |
| BCY9963 | 7100 | ~ | ~ |
| BCY9964 | 35 | ~ | ~ |
| BCY9965 | 77.6 | ~ | ~ |
| BCY9966 | 240 | ~ | ~ |
| BCY9968 | 163 | ~ | ~ |
| BCY10223 | 400 | ~ | ~ |
| BCY10224 | 97.8 | ~ | ~ |
| BCY9965 | ~ | 76 | ~ |
| BCY11147 | ~ | 53 | ~ |
| BCY11148 | ~ | 45 | ~ |
| BCY11149 | ~ | 80 | ~ |
| BCY11150 | ~ | 396 | ~ |
| BCY11151 | ~ | 35 | ~ |
| BCY11152 | ~ | 68 | ~ |
| BCY11153 | ~ | 43 | ~ |
| BCY11154 | ~ | 129 | ~ |
| BCY11155 | ~ | 459 | ~ |
| BCY11163 | ~ | 54 | ~ |
| BCY11158 | ~ | 124 | ~ |
| BCY11160 | ~ | ~ | 289 |
| BCY11165 | ~ | ~ | 72 |
| BCY11166 | ~ | ~ | 92 |
| BCY11167 | ~ | ~ | 135 |
| BCY10288 | 52.5 | ~ | ~ |
| BCY12401 | 10.834 | | |
| BCY12402 | 8.9004 | | |
| BCY12403 | 56.125 | | |
| BCY12404 | 27.44 | | |
| BCY12405 | 14.7 | | |

In Vivo Efficacy Test of BT17BDC58 in Treatment of HT1080 Xenograft in BALB/c Nude Mice

### 1. STUDY OBJECTIVE

The objective of this study was to evaluate the in vivo anti-tumor efficacy of BT17BDC58 in the treatment of HT1080 xenograft model in BALB/c nude mice.

### 2. EXPERIMENTAL DESIGN

| | | | | Dosing | | |
|---|---|---|---|---|---|---|
| | | Dosage | | Volume | Dosing | |
| Gr | Treatment | (mg/kg) | n | (ml/g) | Route | Schedule |
| 1 | Vehicle | — | 3 | 10 | i.v. | biw*2 weeks |
| 2 | BT17BDC58 | 1 | 3 | 10 | i.v. | biw*2 weeks |
| 3 | BT17BDC58 | 3 | 3 | 10 | i.v. | biw*2 weeks |
| 4 | BT17BDC58 | 10 | 3 | 10 | i.v. | biw*2 weeks |

Note:
n: animal number; Dosing volume: adjust dosing volume based on body weight 10 μl/g.

### 3. MATERIALS

3.1 Animals and Housing Condition

3.1.1. Animals
Species: Mus Musculus
Strain: Balb/c nude
Age: 6-8 weeks
Sex: female
Body weight: 18-22 g
Number of animals: 21 mice plus spare
Animal supplier: Shanghai LC Laboratory Animal Co., LTD.

3.1.2. Housing Condition
The mice were kept in individual ventilation cages at constant temperature and humidity with 3 animals in each cage.
   Temperature: 20-26° C.
   Humidity 40-70%.
Cages: Made of polycarbonate. The size is 300 mm×180 mm×150 mm. The bedding material is corn cob, which is changed twice per week.
Diet: Animals had free access to irradiation sterilized dry granule food during the entire study period.
Water: Animals had free access to sterile drinking water.
Cage identification: The identification labels for each cage contained the following information: number of animals, sex, strain, the date received, treatment, study number, group number and the starting date of the treatment.
Animal identification: Animals were marked by ear coding.

3.2 Test and Positive Control Articles
Product identification: BT17BDC58
Manufacturer: Bicycle Therapeutics
Lot number: 1
Physical description: Lyophilised powder
Molecular weight: 7.6 mg
Purity: 98.36%
Package and storage condition: stored at −80° C.

### 4. EXPERIMENTAL METHODS AND PROCEDURES

4.1 Cell Culture

The HT1080 tumor cells were maintained in vitro as a monolayer culture in medium supplemented with 10% heat inactivated fetal bovine serum at 37° C. in an atmosphere of 5% CO₂ in air. The tumor cells were routinely subcultured twice weekly by trypsin-EDTA treatment. The cells growing in an exponential growth phase were harvested and counted for tumor inoculation.

4.2 Tumor Inoculation

Each mouse was inoculated subcutaneously at the right flank with HT1080 tumor cells (5×10⁶) in 0.2 ml of PBS for tumor development. 21 animals were randomized when the average tumor volume reached 174 mm³. The test article administration and the animal numbers in each group were shown in the experimental design table.

4.3 Testing Article Formulation Preparation

| | Dose | |
|---|---|---|
| Treatment | (mg/ml) | Formulation |
| Vehicle | — | 25 mM Histidine pH 7.0, 10% Sucrose (without |
| | | DMSO) |
| BT17BDC58 | 1 | Dissolve 7.6 mg BT17BDC58 into 7.475 ml |
| | | formulation buffer |
| BT17BDC58 | 0.3 | Dilute 240 μl 1 mg/ml BT17BDC58 into 560 μl |
| | | formulation buffer |
| BT17BDC58 | 0.1 | Dilute 80 μl 1 mg/ml BT17BDC58 into 720 μl |
| | | formulation buffer |

4.4 Observations

All the procedures related to animal handling, care and the treatment in the study were performed according to the guidelines approved by the Institutional Animal Care and Use Committee (IACUC) of WuXi AppTec, following the guidance of the Association for Assessment and Accreditation of Laboratory Animal Care (AAALAC). At the time of routine monitoring, the animals were daily checked for any effects of tumor growth and treatments on normal behavior such as mobility, food and water consumption (by looking only), body weight gain/loss, eye/hair matting and any other abnormal effect as stated in the protocol. Death and observed clinical signs were recorded on the basis of the numbers of animals within each subset.

4.5 Tumor Measurements and the Endpoints

The major endpoint was to see if the tumor growth could be delayed or mice could be cured. Tumor volume was measured three times weekly in two dimensions using a caliper, and the volume was expressed in mm³ using the formula: V=0.5 a×b² where a and b are the long and short diameters of the tumor, respectively. The tumor size was then used for calculations of T/C value. The T/C value (in percent) is an indication of antitumor effectiveness; T and C are the mean volumes of the treated and control groups, respectively, on a given day.

TGI was calculated for each group using the formula: TGI (%)=[1−(Ti−T0)/(Vi−V0)]×100; Ti is the average tumor volume of a treatment group on a given day, T0 is the average tumor volume of the treatment group on the day of treatment start, Vi is the average tumor volume of the vehicle control group on the same day with Ti, and V0 is the average tumor volume of the vehicle group on the day of treatment start.

4.6 Sample Collection

At the end of the study, plasma was collected at 5 min, 15 min, 30 min, 60 min and 120 min post dosing.

4.7 Statistical Analysis

Summary statistics, including mean and the standard error of the mean (SEM), are provided for the tumor volume of each group at each time point.

Statistical analysis of difference in tumor volume among the groups was conducted on the data obtained at the best therapeutic time point after the final dose.

A one-way ANOVA was performed to compare tumor volume among groups, and when a significant F-statistics (a ratio of treatment variance to the error variance) was obtained, comparisons between groups were carried out with Games-Howell test. All data were analyzed using Prism. P<0.05 was considered to be statistically significant.

### 5. RESULTS

5.1 Body Weight Change and Tumor Growth Curve

Body weight and tumor growth are shown in FIG. 1.

5.2 Tumor Volume Trace

Mean tumor volume over time in female Balb/c nude mice bearing HT1080 xenograft is shown in Table 4.

TABLE 4
Tumor volume trace over time

| | | Days after the start of treatment | | | | | | |
|---|---|---|---|---|---|---|---|---|
| Gr. | Treatment | 0 | 2 | 4 | 7 | 9 | 11 | 14 |
| 1 | Vehicle, biw | 174 ± 18 | 318 ± 30 | 473 ± 31 | 688 ± 87 | 859 ± 148 | 975 ± 167 | 1075 ± 164 |
| 2 | BT17BDC58, | 174 ± 22 | 265 ± 46 | 351 ± 69 | 488 ± 75 | 577 ± 62 | 676 ± 79 | 785 ± 58 |
| | 1 mpk, biw | | | | | | | |
| 3 | BT17BDC58, | 174 ± 25 | 151 ± 16 | 73 ± 19 | 42 ± 11 | 50 ± 4 | 67 ± 9 | 128 ± 16 |
| | 3 mpk, biw | | | | | | | |
| 4 | BT17BDC58, | 173 ± 25 | 153 ± 29 | 58 ± 20 | 27 ± 1 | 18 ± 4 | 10 ± 1 | 2 ± 2 |
| | 10 mpk, biw | | | | | | | |

5.3 Tumor Growth Inhibition Analysis

Tumor growth inhibition rate for BT17BDC58 in the HT1080 xenograft model was calculated based on tumor volume measurements at day 14 after the start of treatment.

TABLE 5
Tumor growth inhibition analysis

| | | Tumor | | | |
|---|---|---|---|---|---|
| | | Volume | T/C^{b} | TGI | |
| Gr | Treatment | (mm³)^{a} | (%) | (%) | P value |
| 1 | Vehicle, biw | 1075 ± 164 | — | — | — |
| 2 | BT17BDC58, | 785 ± 58 | 73 | 32 | p > 0.05 |
| | 1 mpk, biw | | | | |
| 3 | BT17BDC58, | 128 ± 16 | 12 | 105 | p < 0.001 |
| | 3 mpk, biw | | | | |
| 4 | BT17BDC58, | 2 ± 2 | 0.2 | 119 | p < 0.001 |
| | 10 mpk, biw | | | | |

^{a}Mean ± SEM.
^{b}Tumor Growth Inhibition is calculated by dividing the group average tumor volume for the treated group by the group average tumor volume for the control group (T/C).

### 6. RESULTS SUMMARY AND DISCUSSION

In this study, the therapeutic efficacy of BT17BDC58 in the HT1080 xenograft model was evaluated. The measured body weights and tumor volumes of all treatment groups at various time points are shown in FIG. 1 and Tables 4 and 5.

The mean tumor size of vehicle treated mice reached 1075 mm³ on day 14.

BT17BDC58 at 1 mg/kg (TV=785 mm³, TGI=32.2%, p>0.05), 3 mg/kg (TV=128 mm³, TGI=105.1%, p<0.001) and 10 mg/kg (TV=2 mm³, TGI=84.3%, p<0.001) produced dose-dependent antitumor activity. Among them, BT17BDC58 at 10 mg/kg caused complete remission of ⅔ tumors and regressed ⅓ tumor to 7 mm³ on day 14.

1. A peptide ligand specific for MT1-MMP comprising a polypeptide comprising at least three cysteine residues, separated by at least two loop sequences, and a molecular scaffold which forms covalent bonds with the cysteine residues of the polypeptide such that at least two polypeptide loops are formed on the molecular scaffold, characterised in that said molecular scaffold is 1,1,1″-(1,3,5-triazinane-1,3,5-triyl)triprop-2-en-1-one (TATA).

2. The peptide ligand as defined in claim 1, wherein said loop sequences comprise 2, 3, 5, 6, 7 or 9 amino acids, such as 3 or 7 amino acids.

3. The peptide ligand as defined in claim 1 or claim 2, wherein said loop sequences comprise three cysteine residues separated by two loop sequences a first loop which consists of 7 amino acids and a second loop which consists of 2 amino acids, such as:
(SEQ ID NO: 1)
CEESFYPECDHC;
in particular:
A-(SEQ ID NO: 1)-A (herein referred to as 17-108-02).

4. The peptide ligand as defined in claim 1 or claim 2, wherein said loop sequences comprise three cysteine residues separated by two loop sequences a first loop which consists of 3 amino acids and a second loop which consists of 6 amino acids, such as:
(SEQ ID NO: 2)
CPDLCLDLFPNC;
and
(SEQ ID NO: 3)
CPELCVDLYPHC;
in particular:
A-(SEQ ID NO: 2)-A (herein referred to as 17-111-01).
A-(SEQ ID NO: 3)-A (herein referred to as 17-111-02).

5. The peptide ligand as defined in claim 1 or claim 2, wherein said loop sequences comprise three cysteine residues separated by two loop sequences a first loop which consists of 6 amino acids and a second loop which consists of 3 amino acids, such as:
(SEQ ID NO: 4)
CHPEWVSCEFHC;
in particular:
A-(SEQ ID NO: 4)-A (herein referred to as 17-116-01).

6. The peptide ligand as defined in claim 1 or claim 2, wherein said loop sequences comprise three cysteine residues separated by two loop sequences a first loop which consists of 3 amino acids and a second loop which consists of 7 amino acids, such as
(SEQ ID NO: 5)
CSHECALLFPKTC;
(SEQ ID NO: 6)
CFDECQLLFPKTC;
(SEQ ID NO: 7)
CLDECKLLFPKTC;
(SEQ ID NO: 8)
CREECMLLFPKTC;
(SEQ ID NO: 9)
CETECALLFPRSC;
(SEQ ID NO: 10)
CADECRLLFPKTC;
(SEQ ID NO: 11)
CDVECRLLFPRSC;
(SEQ ID NO: 12)
CIDECRLLFPRSC;
(SEQ ID NO: 13)
CVRECALLFPKTC;
(SEQ ID NO: 14)
CV[HArg]ECALLFPKTC;
(SEQ ID NO: 15)
CVRECALLFPRTC;
(SEQ ID NO: 16)
CVRECALLFP[HArg]TC;
(SEQ ID NO: 17)
CV[HArg]ECALLFP[HArg]TC;
(SEQ ID NO: 18)
CV[HArg]ECALLFPATC;
(SEQ ID NO: 19)
CVAECALLFP[HArg]TC;
(SEQ ID NO: 20)
CVTECQLLFPKTC;
(SEQ ID NO: 21)
CRHECELLFPKTC;
(SEQ ID NO: 22)
CQRECALLFPKTC;
(SEQ ID NO: 23)
CVRECTLLFPKTC;
(SEQ ID NO: 24)
CTIECALLFPKTC;
(SEQ ID NO: 25)
CARECALLFPKTC;
(SEQ ID NO: 26)
CINECRLLFPKTC;
(SEQ ID NO: 27)
CYTECSLLFPKTC;
(SEQ ID NO: 28)
CHEECRLLFPKTC;
(SEQ ID NO: 29)
CLEECKLLFPKTC;
(SEQ ID NO: 30)
CIDECALLFPRTC;
(SEQ ID NO: 31)
CYEECRLLFPRTC;
(SEQ ID NO: 32)
CVRECRLLFPKTC;
(SEQ ID NO: 33)
CHIECALLFPKTC;
(SEQ ID NO: 34)
CKRECMLLFPKTC;
(SEQ ID NO: 35)
CYRECALLFPKTC;
(SEQ ID NO: 36)
CLTECALLFPKTC;
(SEQ ID NO: 37)
CEVECRLLFPKTC;
(SEQ ID NO: 38)
CEAECRLLFPKTC;
(SEQ ID NO: 39)
CVQECALLFPKTC;
(SEQ ID NO: 40)
CIRECSLLFPKTC;
(SEQ ID NO: 41)
CVTECALLFPKTC;
(SEQ ID NO: 42)
CVAECKLLFPKTC;
(SEQ ID NO: 43)
CVGECALLFPKTC;
(SEQ ID NO: 44)
CVVECALLFPKTC;
(SEQ ID NO: 45)
CVFECALLFPKTC;
(SEQ ID NO: 46)
CA[HArg]ECALLFP[HArg]TC;
(SEQ ID NO: 47)
CV[HArg]ECALLFA[HArg]TC;
(SEQ ID NO: 48)
CV[HArg]ECALLFP[HArg]AC;
(SEQ ID NO: 49)
CV[HArg]ECALL[1Nal]P[HArg]TC;
(SEQ ID NO: 50)
CV[HArg]ECALL[Cha]P[HArg]TC;
(SEQ ID NO: 51)
CV[HArg]ECALLF[Pip][HArg]TC;
(SEQ ID NO: 52)
CV[HArg]ECALLFP[HArg]SC;
(SEQ ID NO: 53)
CV[HArg]ECALLFP[HArg][HSer]C;
(SEQ ID NO: 54)
CV[HArg]ECALLF[HyP][HArg]TC;
(SEQ ID NO: 55)
CV[HArg]EC[Aib]LLFP[HArg]TC;
(SEQ ID NO: 56)
CV[HArg]ECAL[Nle]FP[HArg]TC;
(SEQ ID NO: 57)
CV[HArg]ECA[tBuAla]LFP[HArg]TC;
(SEQ ID NO: 58)
CV[HArg]ECA[Nle]LFP[HArg]TC;
(SEQ ID NO: 59)
CV[Aad2]ECALLFP[HArg]TC;
(SEQ ID NO: 60)
CP[HArg]ECALLFP[HArg]TC;
(SEQ NO: 61)
CV[HArg]ECALL[4F1Phe]P[HArg]TC;
(SEQ ID NO: 62)
CV[HArg]ECAL[tBuGly]FP[HArg]TC;
(SEQ ID NO: 63)
CV[HArg]ECAL[Cha]FP[HArg]TC;
(SEQ ID NO: 64)
CV[HArg]ECALL[2Nal]P[HArg]TC;
(SEQ ID NO: 65)
CV[HArg]ECALLFP[HArg][HyV]C;
(SEQ ID NO: 66)
C[tBuGly][HArg]ECALLFP[HArg]TC;
(SEQ ID NO: 67)
CVEECALLFP[HArg]TC;
(SEQ ID NO: 68)
CV[HArg]ECA[Cpa]LFP[HArg]TC;
(SEQ ID NO: 69)
CV[HArg]ECA[Cba]LFP[HArg]TC;
(SEQ ID NO: 70)
CV[HArg]ECA[C5A]LFP[HArg]TC;
(SEQ ID NO: 71)
CV[HArg]ECA[Cha]LFP[HArg]TC;
(SEQ ID NO: 72)
CV[HArg]ECA[tBuGly]LFP[HArg]TC;
(SEQ ID NO: 73)
CV[HArg]ECALLF[Cis-HyP][HArg]TC;
(SEQ ID NO: 74)
CV[HArg]ECAL[Cpa]FP[HArg]TC;
(SEQ ID NO: 75)
CV[HArg]ECAL[C5A]FP[HArg]TC;
(SEQ ID NO: 76)
CV[HArg]ECA[tBuAla]LF[HyP][HArg]TC;
(SEQ ID NO: 77)
CV[HArg]ECA[tBuAla][tBuGly]F[Hylp][HArg]TC;
and
(SEQ ID NO: 78)
C[tBuGly][HArg]ECA[tBuAla]LFP[HArg]TC;
wherein Aad represents alpha-L-aminoadipic acid, Aib represents aminoisobutyric acid, C5a represents beta-cyclopentyl-L-alanine, Cba represents β-cyclobutylalanine, Cha represents 3-cyclohexyl-L-alanine, Cpa represents beta-cyclopropyl-L-alanine, 4FIPhe represents 4-fluoro-L-phenylalanine, HArg represents homoarginine, HyP represents hydroxyproline, HyV represents 3-hydroxy-L-valine, HSer represents homoserine, 1Nal represents 1-naphthylalanine, 2Nal represents 2-naphthylalanine, Ne represents norleucine, Pip represents pipecolic acid, tBuAla represents t-butyl-alanine, tBuGly represents t-butyl-glycine;
in particular:
A-(SEQ ID NO: 5)-A (herein referred to as 17-120-00);
A-(SEQ ID NO: 6)-A (herein referred to as 17-120-01);
A-(SEQ ID NO: 7)-A (herein referred to as 17-120-02);
A-(SEQ ID NO: 8)-A (herein referred to as 17-120-03);
A-(SEQ ID NO: 9)-A (herein referred to as 17-120-04);
A-(SEQ ID NO: 10)-A (herein referred to as 17-120-05);
A-(SEQ ID NO: 11)-A (herein referred to as 17-120-07);
A-(SEQ ID NO: 12)-A (herein referred to as 17-120-08);
APPP-(SEQ ID NO: 13)-A (herein referred to as 17-120-09-T01);
QISP-(SEQ ID NO: 13)-A (herein referred to as 17-120-09-T02);
ALPP-(SEQ ID NO: 13)-A (herein referred to as 17-120-09-T03 and BCY1124);
Ac-ALPP-(SEQ ID NO: 13) (herein referred to as Ac-(17-120-09-T03) and BCY1125);
Sar3-ALPP-(SEQ ID NO: 13) (herein referred to as Sar3-A-(17-120-09-T03));
GPPP-(SEQ ID NO: 13)-A (herein referred to as 17-120-09-T04);
SPPP-(SEQ ID NO: 13)-A (herein referred to as 17-120-09-T05);
NPPP-(SEQ ID NO: 13)-A (herein referred to as 17-120-09-T06);
EPPP-(SEQ ID NO: 13)-A (herein referred to as 17-120-09-T07);
HPPP-(SEQ ID NO: 13)-A (herein referred to as 17-120-09-T08);
APNP-(SEQ ID NO: 13)-A (herein referred to as 17-120-09-T09);
APDP-(SEQ ID NO: 13)-A (herein referred to as 17-120-09-T10);
APLP-(SEQ ID NO: 13)-A (herein referred to as 17-120-09-T11);
APAP-(SEQ ID NO: 13)-A (herein referred to as 17-120-09-T12);
APHP-(SEQ ID NO: 13)-A (herein referred to as 17-120-09-T13);
Sar3-ALPP-(SEQ ID NO: 14) (herein referred to as Sar3-A-(17-120-09-T03) HArg2);
Sar3-ALPP-(SEQ ID NO: 15) (herein referred to as Sar3-A-(17-120-09-T03) Arg9);
Sar3-ALPP-(SEQ ID NO: 16) (herein referred to as Sar3-A-(17-120-09-T03) HArg9);
(B-Ala)-Sar10-ALPP-(SEQ ID NO: 17) (herein referred to as (B-Ala)-Sar10-A-(17-120-09-T03) HArg2 HArg9);
Ac-(B-Ala)-Sar10-ALPP-(SEQ ID NO: 17) (herein referred to as Ac-(B-Ala)-Sar10-A-(17-120-09-T03) HArg2 HArg9);
ALPP-(SEQ ID NO: 17) (herein referred to as BCY3959);
[Ac]LPP-(SEQ ID NO: 17) (herein referred to as BCY9933);
[Ac]APP-(SEQ ID NO: 17) (herein referred to as BCY9934);
[Ac]LAP-(SEQ ID NO: 17) (herein referred to as BCY9935);
[Ac]LPA-(SEQ ID NO: 17) (herein referred to as BCY9936);
[Ac]-(SEQ ID NO: 17) (herein referred to as BCY9968);
[Ac]LYP-(SEQ ID NO: 17) (herein referred to as BCY11147);
[Ac]LPY-(SEQ ID NO: 17) (herein referred to as BCY11148);
[Ac][dA]PP-(SEQ ID NO: 17) (herein referred to as BCY11165);
[Ac]L[dA]P-(SEQ ID NO: 17) (herein referred to as BCY11166);
[Ac]LP[dA]-(SEQ ID NO: 17) (herein referred to as BCY11167);
ALPP-(SEQ ID NO: 17)-A (herein referred to as BCY10288);
(B-Ala)-Sar10-ALPP-(SEQ ID NO: 18) (herein referred to as (B-Ala)-Sar10-A-(17-120-09-T03) HArg2 Ala9);
(B-Ala)-Sar10-ALPP-(SEQ ID NO: 19) (herein referred to as (B-Ala)-Sar10-A-(17-120-09-T03) Ala2 HArg9);
[Ac]LPP-(SEQ ID NO: 19) (herein referred to as BCY9938);
APMP-(SEQ ID NO: 20)-A (herein referred to as 17-120-10-T01);
APSP-(SEQ ID NO: 21)-A (herein referred to as 17-120-11-T01);
AALP-(SEQ ID NO: 22)-A (herein referred to as 17-120-12-T01);
ALDP-(SEQ ID NO: 23)-A (herein referred to as 17-120-13-T01);
ADRP-(SEQ ID NO: 24)-A (herein referred to as 17-120-14-T01);
ATQP-(SEQ ID NO: 25)-A (herein referred to as 17-120-15-T01);
SPPP-(SEQ ID NO: 25)-A (herein referred to as 17-120-15-T02);
ARHP-(SEQ ID NO: 26)-A (herein referred to as 17-120-16-T01);
ALPP-(SEQ ID NO: 27)-A (herein referred to as 17-120-17-T01);
A-(SEQ ID NO: 28)-A (herein referred to as 17-120-18);
A-(SEQ ID NO: 29)-A (herein referred to as 17-120-19);
A-(SEQ ID NO: 30)-A (herein referred to as 17-120-20);
A-(SEQ ID NO: 31)-A (herein referred to as 17-120-21);
APPP-(SEQ ID NO: 31)-A (herein referred to as 17-120-21-T01);
APSP-(SEQ ID NO: 32)-A (herein referred to as 17-120-22-T01);
PLPP-(SEQ ID NO: 32)-A (herein referred to as 17-120-22-T02);
APAP-(SEQ ID NO: 33)-A (herein referred to as 17-120-23-T01);
AVEP-(SEQ ID NO: 34)-A (herein referred to as 17-120-24-T01);
AEPA-(SEQ ID NO: 35)-A (herein referred to as 17-120-25-T01);
ASPP-(SEQ ID NO: 36)-A (herein referred to as 17-120-26-T01);
AAPP-(SEQ ID NO: 37)-A (herein referred to as 17-120-27-T01);
APPP-(SEQ ID NO: 38)-A (herein referred to as 17-120-28-T01);
AVPP-(SEQ ID NO: 39)-A (herein referred to as 17-120-29-T01);
SPPP-(SEQ ID NO: 40)-A (herein referred to as 17-120-30-T01);
HLPP-(SEQ ID NO: 41)-A (herein referred to as 17-120-31-T01);
RLPP-(SEQ ID NO: 41)-A (herein referred to as 17-120-31-T02);
APPP-(SEQ ID NO: 41)-A (herein referred to as 17-120-31-T03);
MPPP-(SEQ ID NO: 42)-A (herein referred to as 17-120-32-T01);
SPPP-(SEQ ID NO: 43)-A (herein referred to as 17-120-33-T01);
APPP-(SEQ ID NO: 44)-A (herein referred to as 17-120-34-T01);
APPP-(SEQ ID NO: 45)-A (herein referred to as 17-120-35-T01);
[Ac]LPP-(SEQ ID NO: 46) (herein referred to as BCY9937);
[Ac]LPP-(SEQ ID NO: 47) (herein referred to as BCY9943);
[Ac]LPP-(SEQ ID NO: 48) (herein referred to as BCY9945);
[Ac]LPP-(SEQ ID NO: 49) (herein referred to as BCY9946);
[Ac]LPP-(SEQ ID NO: 50) (herein referred to as BCY9949);
[Ac]LPP-(SEQ ID NO: 51) (herein referred to as BCY9951);
[Ac]LPP-(SEQ ID NO: 52) (herein referred to as BCY9952);
[Ac]LPP-(SEQ ID NO: 53) (herein referred to as BCY9953);
[Ac]LPP-(SEQ ID NO: 54) (herein referred to as BCY9954);
[Ac]LPP-(SEQ ID NO: 55) (herein referred to as BCY9955);
[Ac]LPP-(SEQ ID NO: 56) (herein referred to as BCY9957);
[Ac]LPP-(SEQ ID NO: 57) (herein referred to as BCY9959);
[Ac]LYP-(SEQ ID NO: 57) (herein referred to as BCY12401);
[Ac]EYP-(SEQ ID NO: 57) (herein referred to as BCY12405);
[Ac]LPP-(SEQ ID NO: 58) (herein referred to as BCY9960);
[Ac]LPP-(SEQ ID NO: 59) (herein referred to as BCY9961);
[Ac]LPP-(SEQ ID NO: 60) (herein referred to as BCY9963);
[Ac]LPP-(SEQ ID NO: 61) (herein referred to as BCY9964);
[Ac]LPP-(SEQ ID NO: 62) (herein referred to as BCY9965);
[Ac]LPP-(SEQ ID NO: 63) (herein referred to as BCY9966);
[Ac]LPP-(SEQ ID NO: 64) (herein referred to as BCY10223);
[Ac]LPP-(SEQ ID NO: 65) (herein referred to as BCY10224);
[Ac]LPP-(SEQ ID NO: 66) (herein referred to as BCY11149);
[Ac]LPP-(SEQ ID NO: 67) (herein referred to as BCY11150);
[Ac]LPP-(SEQ ID NO: 68) (herein referred to as BCY11151);
[Ac]LPP-(SEQ ID NO: 69) (herein referred to as BCY11152);
[Ac]LPP-(SEQ ID NO: 70) (herein referred to as BCY11153);
[Ac]LPP-(SEQ ID NO: 71) (herein referred to as BCY11154);
[Ac]LPP-(SEQ ID NO: 72) (herein referred to as BCY11155);
[Ac]LPP-(SEQ ID NO: 73) (herein referred to as BCY11163);
[Ac]LPP-(SEQ ID NO: 74) (herein referred to as BCY11158);
[Ac]LPP-(SEQ ID NO: 75) (herein referred to as BCY11160);
[Ac]LYP-(SEQ ID NO: 76) (herein referred to as BCY12402);
[Ac]LYP-(SEQ ID NO: 77) (herein referred to as BCY12403); and
[Ac]LYP-(SEQ ID NO: 78) (herein referred to as BCY12404).

7. The peptide ligand as defined in claim 6, wherein the peptide ligand comprises an amino acid sequence which is (B-Ala)-Sar10-ALPP-(SEQ ID NO: 17) (herein referred to as (B-Ala)-Sar10-A-(17-120-09-T03) HArg2 HArg9).

8. The peptide ligand as defined in claim 1 or claim 2, wherein said loop sequences comprise three cysteine residues separated by two loop sequences a first loop which consists of 7 amino acids and a second loop which consists of 3 amino acids, such as:
(SEQ ID NO: 79)
CSSWDKLMCHPYC;
in particular:
A-(SEQ ID NO: 79)-A (herein referred to as 17-121-00).

9. The peptide ligand as defined in claim 1 or claim 2, wherein said loop sequences comprise three cysteine residues separated by two loop sequences a first loop which consists of 3 amino acids and a second loop which consists of 9 amino acids, such as:
(SEQ ID NO: 80)
CPEECFYLPPHPMSC;
(SEQ ID NO: 81)
CPQECFYLPGHSLYC;
(SEQ ID NO: 82)
CPGECFYPPGHPLAC;
(SEQ ID NO: 83)
CPGECFYPTNHPLYC;
(SEQ ID NO: 84)
CPQECFYPIGHPLAC;
(SEQ ID NO: 85)
CPEECFYPPGHKLHC;
(SEQ ID NO: 86)
CPQECFYPPGHRLRC;
(SEQ ID NO: 87)
CPQECFYPPGHPYHC;
(SEQ ID NO: 88)
CPQECFYPSTHPLYC;
(SEQ ID NO: 89)
CPGECFYPSNHRLYC;
(SEQ ID NO: 90)
CPDECFYPPEHPLAC;
(SEQ ID NO: 91)
CPGECFYPPGHHLSC;
(SEQ ID NO: 92)
CPGECFYPPGHHLGC;
(SEQ ID NO: 93)
CPEECFYPPNHPLYC;
(SEQ ID NO: 94)
CPGECFYPPDHPLYC;
(SEQ ID NO: 95)
CPGECFYPPGHPLYC;
(SEQ ID NO: 96)
CPGECFYPPNHPFYC;
(SEQ ID NO: 97)
CPGECFYPPNHPLYC;
(SEQ ID NO: 98)
CPEECFYPPGHPLAC;
(SEQ ID NO: 99)
CWMECFYPPGHPLAC;
(SEQ ID NO: 100)
CFEECFYPPGHPLAC;
(SEQ ID NO: 101)
CPGECFYPPGHPLRC;
(SEQ ID NO: 102)
CPGECFYPPGHPREC;
(SEQ ID NO: 103)
CPGECFYPPGHRFHC;
and
(SEQ ID NO: 104)
CPGECFYPPGHRLYC;
in particular:
A-(SEQ ID NO: 80)-A (herein referred to as 17-127-01);
A-(SEQ ID NO: 81)-A (herein referred to as 17-129-00);
SQT-(SEQ ID NO: 82)-A (herein referred to as 17-129-01-T01);
SMT-(SEQ ID NO: 82)-A (herein referred to as 17-129-01-T02);
SLV-(SEQ ID NO: 82)-A (herein referred to as 17-129-01-T03);
ISSYG-(SEQ ID NO: 82)-A (herein referred to as 17-129-01-T04);
ENITT-(SEQ ID NO: 82)-A (herein referred to as 17-129-01-T05);
A-(SEQ ID NO: 83)-A (herein referred to as 17-129-02);
A-(SEQ ID NO: 84)-A (herein referred to as 17-129-03);
A-(SEQ ID NO: 85)-A (herein referred to as 17-129-04);
A-(SEQ ID NO: 86)-A (herein referred to as 17-129-05);
A-(SEQ ID NO: 87)-A (herein referred to as 17-129-06);
A-(SEQ ID NO: 88)-A (herein referred to as 17-129-07);
A-(SEQ ID NO: 89)-A (herein referred to as 17-129-08);
A-(SEQ ID NO: 90)-A (herein referred to as 17-129-09);
A-(SEQ ID NO: 91)-A (herein referred to as 17-129-10);
A-(SEQ ID NO: 92)-A (herein referred to as 17-129-11);
L-(SEQ ID NO: 93)-HA (herein referred to as 17-129-12-T01);
T-(SEQ ID NO: 94)-NA (herein referred to as 17-129-13-T01);
Q-(SEQ ID NO: 95)-NA (herein referred to as 17-129-14-T01);
A-(SEQ ID NO: 95)-NVI (herein referred to as 17-129-14-T02);
N-(SEQ ID NO: 96)-NA (herein referred to as 17-129-15-T01);
D-(SEQ ID NO: 97)-RA (herein referred to as 17-129-16-T01);
SRM-(SEQ ID NO: 98)-A (herein referred to as 17-129-17-T01);
SRS-(SEQ ID NO: 98)-A (herein referred to as 17-129-17-T02);
RYMTR-(SEQ ID NO: 98)-A (herein referred to as 17-129-17-T03);
REE-(SEQ ID NO: 99)-A (herein referred to as 17-129-18-T01);
DNM-(SEQ ID NO: 99)-A (herein referred to as 17-129-18-T02);
QES-(SEQ ID NO: 99)-A (herein referred to as 17-129-18-T03);
ADY-(SEQ ID NO: 99)-A (herein referred to as 17-129-18-T04);
MAN-(SEQ ID NO: 100)-A (herein referred to as 17-129-19-T01);
SQN-(SEQ ID NO: 100)-A (herein referred to as 17-129-19-T02);
A-(SEQ ID NO: 101)-TVL (herein referred to as 17-129-20-T01);
A-(SEQ ID NO: 102)-SWL (herein referred to as 17-129-21-T01);
A-(SEQ ID NO: 103)-LTE (herein referred to as 17-129-22-T01);
A-(SEQ ID NO: 104)-YSE (herein referred to as 17-129-23-T01); and
Ac-(SEQ ID NO: 104)-YSE (herein referred to as Ac(17-129-23-T01)).

10. The peptide ligand as defined in claim 1 or claim 2, wherein said loop sequences comprise three cysteine residues separated by two loop sequences a first loop which consists of 6 amino acids and a second loop which consists of 6 amino acids, such as
(SEQ ID NO: 105)
CEEEFYPCGHPLYVC;
(SEQ ID NO: 106)
CEEQFYPCTHALYTC;
(SEQ ID NO: 107)
CVEEFYPCDHPLYSC;
(SEQ ID NO: 108)
CEEEFYPCGHPMHPC;
(SEQ ID NO: 109)
CDEQFYPCHHRLYSC;
(SEQ ID NO: 110)
CEEEFYPCGHPFHPC;
(SEQ ID NO: 111)
CLEQFYPCEHPLFSC;
(SEQ ID NO: 112)
CVEQFYPCGHRHYIC;
(SEQ ID NO: 113)
CEEQFYPCSHPLYTC;
(SEQ ID NO: 114)
CEEQFYPCNHPLNVC;
(SEQ ID NO: 115)
CEEEFYPCSHPLNPC;
(SEQ ID NO: 116)
CEEQFYPCGHKLSPC;
(SEQ ID NO: 117)
CPEQFYPCDHRLYIC;
(SEQ ID NO: 118)
CQEQFYPCNHPLSPC;
(SEQ ID NO: 119)
CDEQFYPCNHRLNTC;
(SEQ ID NO: 120)
CEEAFYPCHHPLYRC;
(SEQ ID NO: 121)
CDEDFYPCGHYLNQC;
(SEQ ID NO: 122)
CEEQFYPCTHPLYVC;
(SEQ ID NO: 123)
CPEQFYPCTHRLYQC;
(SEQ ID NO: 124)
CEEQFYPCSHPLYRC;
(SEQ ID NO: 125)
CAEQFYPCDHPLYRC;
(SEQ ID NO: 126)
CAEEFYPCDHPLYRC;
(SEQ ID NO: 127)
CEEAFYPCNHPLYTC;
(SEQ ID NO: 128)
CAEAFYPCDHPLYVC;
(SEQ ID NO: 129)
CEEAFYPCSHPLFIC;
(SEQ ID NO: 130)
CEEAFYPCSHPLHPC;
(SEQ ID NO: 131)
CEEAFYPCSHPLFVC;
(SEQ ID NO: 132)
CEEQFYPCSHPLYSC;
(SEQ ID NO: 133)
CEEAFYPCEHPLYMC;
and
(SEQ ID NO: 134)
CEEQFYPCNHPLYMC;
in particular:
A-(SEQ ID NO: 105)-A (herein referred to as 17-126-01);
A-(SEQ ID NO: 106)-A (herein referred to as 17-126-02);
A-(SEQ ID NO: 107)-A (herein referred to as 17-126-03);
A-(SEQ ID NO: 108)-A (herein referred to as 17-126-06);
A-(SEQ ID NO: 109)-A (herein referred to as 17-126-07);
A-(SEQ ID NO: 110)-A (herein referred to as 17-126-08);
A-(SEQ ID NO: 111)-A (herein referred to as 17-126-09);
A-(SEQ ID NO: 112)-A (herein referred to as 17-126-10);
A-(SEQ ID NO: 113)-A (herein referred to as 17-126-18);
A-(SEQ ID NO: 114)-A (herein referred to as 17-126-19);
A-(SEQ ID NO: 115)-A (herein referred to as 17-126-20);
A-(SEQ ID NO: 116)-A (herein referred to as 17-126-21);
A-(SEQ ID NO: 117)-A (herein referred to as 17-126-22);
A-(SEQ ID NO: 118)-A (herein referred to as 17-126-23);
A-(SEQ ID NO: 119)-A (herein referred to as 17-126-24);
A-(SEQ ID NO: 120)-A (herein referred to as 17-126-25);
Ac-A-(SEQ ID NO: 120)-A (herein referred to as Ac-(17-126-25));
A-(SEQ ID NO: 121)-A (herein referred to as 17-126-26);
A-(SEQ ID NO: 122)-A (herein referred to as 17-126-27);
A-(SEQ ID NO: 123)-A (herein referred to as 17-126-28);
HSP-(SEQ ID NO: 124)-A (herein referred to as 17-126-30-T01);
GPH-(SEQ ID NO: 125)-A (herein referred to as 17-126-31-T01);
IHS-(SEQ ID NO: 126)-A (herein referred to as 17-126-32-T01);
WSP-(SEQ ID NO: 127)-A (herein referred to as 17-126-33-T01);
SHS-(SEQ ID NO: 127)-A (herein referred to as 17-126-33-T02);
DLH-(SEQ ID NO: 128)-A (herein referred to as 17-126-35-T01);
ANE-(SEQ ID NO: 129)-A (herein referred to as 17-126-36-T01);
AVW-(SEQ ID NO: 130)-A (herein referred to as 17-126-37-T01);
KVQ-(SEQ ID NO: 131)-A (herein referred to as 17-126-38-T01);
A-(SEQ ID NO: 132)-PDVA (herein referred to as 17-126-39-T01);
A-(SEQ ID NO: 133)-HQAA (herein referred to as 17-126-40-T01); and
A-(SEQ ID NO: 134)-RENA (herein referred to as 17-126-41-T01).

11. The peptide ligand as defined in claim 1 or claim 2, wherein said loop sequences comprise three cysteine residues separated by two loop sequences a first loop which consists of 6 amino acids and a second loop which consists of 5 amino acids, such as:
(SEQ ID NO: 135)
CLEQFYPCGDPRLC;
and
(SEQ ID NO: 136)
CEEQFYPCGHHLLC;
in particular:
A-(SEQ ID NO: 135)-A (herein referred to as 17-126-11); and
A-(SEQ ID NO: 136)-A (herein referred to as 17-126-12).

12. The peptide ligand as defined in claim 1 or claim 2, wherein said loop sequences comprise three cysteine residues separated by two loop sequences a first loop which consists of 5 amino acids and a second loop which consists of 5 amino acids, such as:
(SEQ ID NO: 137)
CLEPDECFYPMEC;
(SEQ ID NO: 138)
CKEPQECFYPLKC;
and
(SEQ ID NO: 139)
CDSPEECFYPLEC;
in particular:
A-(SEQ ID NO: 137)-A (herein referred to as 17-122-02);
A-(SEQ ID NO: 138)-A (herein referred to as 17-122-03); and
A-(SEQ ID NO: 139)-A (herein referred to as 17-122-04).

13. The peptide ligand as defined in claim 1 or claim 2, which is selected from any of the peptide ligands listed in Table 2 or Table 3.

14. The peptide ligand as defined in any one of claims 1 to 13, wherein the pharmaceutically acceptable salt is selected from the free acid or the sodium, potassium, calcium, ammonium salt.

15. The peptide ligand as defined in any one of claims 1 to 14, wherein the MT1-MMP is human MT1-MMP.

16. A drug conjugate comprising a peptide ligand as defined in any one of claims 1 to 15, conjugated to one or more effector and/or functional groups.

17. The drug conjugate as defined in claim 16, conjugated to one or more cytotoxic agents.

18. The drug conjugate as defined in claim 17, wherein said cytotoxic agent is selected from MMAE or DM1.

19. The drug conjugate as defined in claim 18, wherein the cytotoxic agent is MMAE and said conjugate additionally comprises a linker selected from: -PABC-Cit-Val-Glutaryl- or -PABC-cyclobutyl-Ala-Cit-βAla-, such as -PABC-Cit-Val-Glutaryl-, wherein PABC represents p-aminobenzylcarbamate.

20. The drug conjugate as defined in claim 19, which is BT17BDC58: 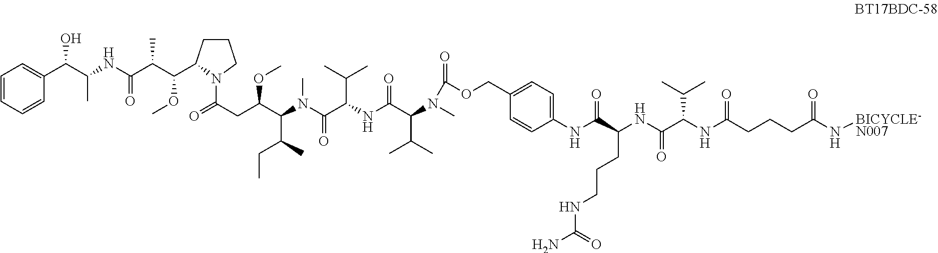
wherein BICYCLE-N007 represents (B-Ala)-Sar10-ALPP-(SEQ ID NO: 17) also known as (B-Ala)-Sar10-A-(17-120-09-T03) HArg2 HArg9.

21. A pharmaceutical composition which comprises the peptide ligand of any one of claims 1 to 15 or the drug conjugate of any one of claims 16 to 20, in combination with one or more pharmaceutically acceptable excipients.

22. The pharmaceutical composition as defined in claim 21, which additionally comprises one or more therapeutic agents.

23. The drug conjugate as defined in any one of claims 16 to 20, for use in preventing, suppressing or treating a disease or disorder mediated by MT1-MMP.
